(19) United States
(12) Patent Application Publication
(10) Pub. No.: US 2020/0365231 A1
(43) Pub. Date: Nov. 19, 2020

(54) INCORPORATION OF FUSION GENES INTO PPI NETWORK TARGET SELECTION VIA GIBBS HOMOLOGY

(71) Applicant: CSTS Health Care Inc., Toronto (CA)

(72) Inventors: Edward A. Rietman, Nashua, NH (US); Giannoula Lakka Klement, Toronto (CA); Ali Hashemi, Toronto (CA)

(73) Assignee: CSTS Health Care Inc., Toronto (CA)

(21) Appl. No.: 16/768,042

(22) PCT Filed: Nov. 28, 2018
(86) PCT No.: PCT/CA2018/051515

§ 371 Date: May 28, 2020

Related U.S. Application Data
(60) Provisional application No. 62/591,572, filed on Nov. 28, 2017.

(51) Int. Cl. G16B 5/20

(52) U.S. Cl. CPC G16B 5/20

(57) ABSTRACT

A method for selecting a molecular target for therapeutic application involves accessing omic information and protein-protein interaction (PPI) data including a network of protein nodes. The method further involves computing a Gibbs free energy for each protein node within the network of protein nodes using the omic information and the PPI data, interpreting information for one or more products of gene fusion from the omic information as one or more gene fusion protein probabilities, and converting the one or more gene fusion protein probabilities into one or more gene fusion protein networks based on a Fermi distribution. The method also involves taking a union of the network of protein nodes with the one or more gene fusion protein networks and generating an energy landscape corresponding to the union of the network of protein nodes with the one or more gene fusion protein networks, and the Gibbs free energy.

### CROSS REFERENCE TO RELATED APPLICATIONS

The present application claims priority under 35 U.S.C. § 119(e) to U.S. Provisional Application No. 62/591,572, filed on Nov. 28, 2017, having at least one of the same inventors as the present application, and entitled, “INCORPORATION OF FUSION GENES INTO PPI NETWORK TARGET SELECTION VIA GIBBS HOMOLOGY”. U.S. Provisional Application No. 62/591,572 is incorporated herein by reference.

### BACKGROUND

As the medical field modernizes and sequencing technology becomes ubiquitous, an increasing amount of online bioinformatics data remains untapped by clinicians for personalized medicine and patient therapy. Bioinformatics data may include human protein-protein interaction (PPI) networks, PPI data generally, patient proteome, whole genome, and transcriptome data. One of the hurdles is that there is a vast volume of patient information being generated through genomics, proteomics and other sources of information, but consolidation is limited due to lack of access, understanding and most importantly lack of tools for appropriate analysis.

It has been established that complexity of cancer PPI networks, as measured by degree-entropy, is strongly correlated with cancer patient survival statistics. However, this kind of statistic does not necessarily include new kinds of proteins that have been created by the fusion of previously unrelated genes. These fusions occur much more frequently in cancer, and many of these fusions result in constitutional activation of genes. The molecular bridges created by fusion proteins can be of key importance in drug and therapy design. Social association of nodes, perturbation centrality, and centrality measures are used to identify important nodes and substrate binding sites and amino acids participating in allosteric signaling in protein structure networks.

### SUMMARY

In general, one or more embodiments relate to a method for selecting a molecular target for therapeutic application, comprising: accessing omic information and protein-protein interaction (PPI) data, the PPI data comprising a network of protein nodes from at least one source; computing a Gibbs free energy for each protein node within the network of protein nodes using the omic information and the PPI data; interpreting information for one or more products of gene fusion from the omic information as one or more gene fusion protein probabilities; converting the one or more gene fusion protein probabilities into one or more gene fusion protein networks based on a Fermi distribution; taking a union of the network of protein nodes with the one or more gene fusion protein networks; and generating an energy landscape data corresponding to the union of the network of protein nodes with the one or more gene fusion protein networks, and the Gibbs free energy.

In general, one or more embodiments relate to non-transitory computer readable medium comprising computer readable program code for causing a computer system to perform operations comprising: accessing omic information and protein-protein interaction (PPI) data, the PPI data comprising a network of protein nodes from at least one source; computing a Gibbs free energy for each protein node within the network of protein nodes using the omic information and the PPI data; interpreting information for one or more products of gene fusion from the omic information as one or more gene fusion protein probabilities; converting the one or more gene fusion protein probabilities into one or more gene fusion protein networks based on a Fermi distribution; taking a union of the network of protein nodes with the one or more gene fusion protein networks; and generating an energy landscape data corresponding to the union of the network of protein nodes with the one or more gene fusion protein networks, and the Gibbs free energy.

In general, one or more embodiments relate to a method for selecting a molecular target for therapeutic application, comprising: accessing omic information and protein-protein interaction (PPI) data, the PPI data comprising a network of protein nodes from at least one source; computing a Gibbs free energy for each protein node within the network of protein nodes using the omic information and the PPI data; interpreting information for one or more products of gene fusion from the omic information as one or more gene fusion protein probabilities; converting the one or more gene fusion protein probabilities into one or more gene fusion protein networks based on a Fermi distribution; interpreting immune regulator information from the omic information as one or more boosted immune regulator weighting values based on a Fermi distribution; taking a union of the network of protein nodes with the one or more gene fusion protein networks and the one or more boosted immune regulator weighting values; generating an energy landscape data corresponding to the union of the network of protein nodes with the one or more gene fusion protein networks and the one or more boosted immune regulator values, and the Gibbs free energy; generating a PPI subnetwork by applying a topological filtration to the energy landscape data; computing at least one of a first Betti number or cycle-basis centrality number for the PPI subnetwork; sequentially removing a first protein node from the PPI subnetwork; computing at least one of a second Betti number or cycle-basis centrality number for the PPI subnetwork with the first protein node removed; computing a change between the first Betti number or cycle-basis centrality number and the second Betti number or cycle-basis centrality number; replacing the first protein node into the PPI subnetwork; sequentially removing a second protein node from the PPI subnetwork, wherein the second protein node is different from the first protein node; computing a third Betti number or cycle-basis centrality number for the PPI subnetwork with the second protein node removed and the first protein node replaced; computing a change between the first Betti number or cycle-basis centrality number and the third Betti number or cycle-basis centrality number; and determining, based on the change between the first Betti number or cycle-basis centrality number and the second Betti number or cycle-basis centrality number and the change between the first Betti number or cycle-basis centrality number and the third Betti number or cycle-basis centrality number, a most significant molecular target within the PPI subnetwork.

One or more embodiments further relate to displaying the most significant molecular targets to a user.

One or more embodiments further relate to storing the omic information and the PPI data in one or more data repositories.

In one or more embodiments, the method further comprises computing one or more additional Betti numbers or cycle-basis centrality numbers for the PPI subnetwork; and wherein the determining the most significant molecular target within the PPI subnetwork further comprises selecting most significant molecular target based on the largest change from all available Betti numbers or cycle-basis centrality numbers.

In one or more embodiments, the omic information is derived from one or more selected from a group consisting of messenger RNA (mRNA), RNA sequencing (RNA-seq), clustered regularly interspaced short palindromic repeats (CRISPR), and mass-spec proteomics.

In one or more embodiments, the Gibbs free energy for each of the protein nodes within the PPI data is computed using the omic information and an equation of:

$${G}_{i} = ( {c}_{i} + {E}_{α}^{( i )} ) ln ( \frac{{c}_{i} + {E}_{α}^{( i )}}{{\sum }_{j = i} ( {c}_{j} + {E}_{α}^{( j )} )} )$$

and an overall Gibbs free energy of all of the protein nodes within the PPI data is computed using an equation of:

$$qG = {\sum }_{i} {G}_{i} .$$

In one or more embodiments, the PPI subnetwork is a persistent homology that is extracted from the energy landscape of the PPI data using the topological filtration based on a user set threshold.

In one or more embodiments, the user set threshold is between 1 to 20,000.

In one or more embodiments, the Betti number or cycle-basis centrality number of the PPI subnetwork is computed based on the number of rings of four or more proteins nodes within the PPI subnetwork.

In one or more embodiments, the Betti number or cycle-basis centrality numbers and removed protein nodes are stored in an array.

In one or more embodiments, the change in the Betti number or cycle-basis centrality number represents an effect that the single protein node has on a network complexity of the PPI data and the single removed protein node that causes a highest drop of the network complexity is the most significant molecular target.

In general, one or more embodiments relate to a method for selecting a molecular target for therapeutic application, comprising: accessing omic information and protein-protein interaction (PPI) data, the PPI data comprising a network of protein nodes from at least one source; computing a Gibbs free energy for each protein node within the network of protein nodes using the omic information and the PPI data; interpreting information for one or more products of gene fusion from the omic information as one or more gene fusion protein probabilities; converting the one or more gene fusion protein probabilities into one or more gene fusion protein networks based on a Fermi distribution; taking a union of the network of protein nodes with the one or more gene fusion protein networks; generating an energy landscape data corresponding to the union of the network of protein nodes with the one or more gene fusion protein networks and the Gibbs free energy; generating a PPI subnetwork by applying a topological filtration to the energy landscape data; computing at least one of a first Betti number or cycle-basis centrality number for the PPI subnetwork; sequentially removing a first protein node from the PPI subnetwork; computing at least one of a second Betti number or cycle-basis centrality number for the PPI subnetwork with the first protein node removed; computing a change between the first Betti number or cycle-basis centrality number and the second Betti number or cycle-basis centrality number; replacing the first protein node into the PPI subnetwork; sequentially removing a second protein node from the PPI subnetwork, wherein the second protein node is different from the first protein node; computing a third Betti number or cycle-basis centrality number for the PPI subnetwork with the second protein node removed and the first protein node replaced; computing a change between the first Betti number or cycle-basis centrality number and the third Betti number or cycle-basis centrality number; determining, based on the change between the first Betti number or cycle-basis centrality number and the second Betti number or cycle-basis centrality number and the change between the first Betti number or cycle-basis centrality number and the third Betti number or cycle-basis centrality number, a most significant molecular target within the PPI subnetwork.

One or more embodiments further relate to displaying the most significant molecular targets to a user.

One or more embodiments further relate to storing the omic information and the PPI data one or more data repositories.

In one or more embodiments, converting the one or more gene fusion protein probabilities into one or more gene fusion protein networks based on a Fermi distribution comprises placing a gene fusion protein on a higher energy level of the Fermi distribution that corresponds with the respective gene fusion probability.

One or more embodiments further relate to interpreting immune regulator information from the omic information as one or more boosted immune regulator weighting values based on a Fermi distribution; wherein taking a union of the network of protein nodes with the one or more gene fusion protein networks further comprises: taking a union of the network of protein nodes with the one or more gene fusion protein networks and the one or more boosted immune regulator weighting values; wherein generating an energy landscape data corresponding to the union of the network of protein nodes with the one or more gene fusion protein networks, and the Gibbs free energy further comprises: generating an energy landscape data corresponding to the union of the network of protein nodes with the one or more gene fusion protein networks and the one or more boosted immune regulator values, and the Gibbs free energy.

In one or more embodiments, Gibbs free energy for each of the protein nodes within the PPI data is computed using the omic information and an equation of:

$${G}_{i} = ( {c}_{i} + {E}_{α}^{( i )} ) ln ( \frac{{c}_{i} + {E}_{α}^{( i )}}{{\sum }_{j = i} ( {c}_{j} + {E}_{α}^{( j )} )} )$$

and an overall Gibbs free energy of all of the protein nodes within the PPI data is computed using an equation of:

$$qG = {\sum }_{i} {G}_{i} .$$

In general, one or more embodiments relate to non-transitory computer readable medium comprising computer readable program code for causing a computer system to perform operations comprising: accessing omic information and protein-protein interaction (PPI) data, the PPI data comprising a network of protein nodes from at least one source; computing, using the omic information and the PPI data, a Gibbs free energy for each protein node within the network of protein nodes; interpreting genomic fusion information from the omic information as one or more genomic fusion protein probabilities; converting the genomic fusion protein probabilities into a set genomic protein fusion networks based on a Fermi distribution; assigning a interpreting immune regulators with a boosted weighting value based on a Fermi distribution; taking a union of the network described in step 2 with the fusion networks and/or supplemented by the immune regulator weights; converting the one or more key protein probabilities into one or more key protein networks based on a Fermi distribution; taking a union of the network of protein nodes with the one or more key protein networks; generating an energy landscape data corresponding to union of the network of protein nodes with the one or more key protein networks and the Gibbs free energy; generating a PPI subnetwork by applying a topological filtration to the energy landscape data; computing a first Betti number or cycle-basis centrality number for the PPI subnetwork; sequentially removing a first protein node from the PPI subnetwork; computing a second Betti number or cycle-basis centrality number for the PPI subnetwork with the first protein node removed; computing a change between the first Betti number or cycle-basis centrality number and the second Betti number or cycle-basis centrality number; replacing the first protein node into the PPI subnetwork; sequentially removing a second protein node different from the first protein node from the PPI subnetwork; computing a third Betti number or cycle-basis centrality number for the PPI subnetwork with the second protein node removed and first protein node replaced; computing a change between the first Betti number or cycle-basis centrality number and the third Betti number or cycle-basis centrality number; and determining, based on the change between the first Betti number or cycle-basis centrality number and the second Betti number or cycle-basis centrality number and the change between the first Betti number or cycle-basis centrality number and the third Betti number or cycle-basis centrality number, a most significant molecular target within the PPI subnetwork.

In one or more embodiments, instructions stored on the non-transitory computer readable medium further cause the computer system to perform operations comprising displaying the most significant molecular target to a user.

In one or more embodiments, Gibbs free energy for each of the protein nodes within the PPI data is computed using the transcription data and an equation of:

$${G}_{i} = ( {c}_{i} + {E}_{α}^{( i )} ) ln ( \frac{{c}_{i} + {E}_{α}^{( i )}}{{\sum }_{j = i} ( {c}_{j} + {E}_{α}^{( j )} )} )$$

and an overall Gibbs free energy of all of the protein nodes within the PPI data is computed using an equation of:

$$qG = {\sum }_{i} {G}_{i} .$$

Other aspects of the embodiments will be apparent from the following description and the appended claims.

### BRIEF DESCRIPTION OF DRAWINGS

The present embodiments are illustrated by way of example and are not intended to be limited by the figures of the accompanying drawings.

FIGS. 1, 2, and 3 show a graph in accordance with one or more embodiments.

FIG. 4 shows diagrams in accordance with one or more embodiments of the present disclosure.

FIGS. 5A, 5B, 5C, and 5D show diagrams in accordance with one or more embodiments of the present disclosure.

FIGS. 6A and 6B show a graph in accordance with one or more embodiments of the present disclosure.

FIGS. 7, 8, 9, 10, 11, and 12 show graphs in accordance with one or more embodiments of the present disclosure.

FIGS. 13A and 13B show a computing system in accordance with one or more embodiments of the present disclosure.

FIG. 14 shows a schematic diagram in accordance with one or more embodiments of the present disclosure.

FIGS. 15, 16, 17A, 17B, 18A, 18B, and 18C show flowcharts in accordance with one or more embodiments of the present disclosure.

### DETAILED DESCRIPTION

Specific embodiments disclosed herein will now be described in detail with reference to the accompanying figures. Like elements in the various figures may be denoted by like reference numerals and/or like names for consistency.

The following detailed description is merely exemplary in nature, and is not intended to limit the embodiments disclosed herein or the application and uses of embodiments disclosed herein. Furthermore, there is no intention to be bound by any expressed or implied theory presented in the preceding technical field, background, brief summary or the following detailed description.

In the following detailed description of some embodiments disclosed herein, numerous specific details are set forth in order to provide a more thorough understanding of the various embodiments disclosed herein. However, it will be apparent to one of ordinary skill in the art that the embodiments may be practiced without these specific details. In other instances, well-known features have not been described in detail to avoid unnecessarily complicating the description.

Throughout the application, ordinal numbers (e.g., first, second, third, etc.) may be used as an adjective for an element (i.e., any noun in the application). The use of ordinal numbers is not to imply or create any particular ordering of the elements nor to limit any element to being only a single element unless expressly disclosed, such as by the use of the terms “before”, “after”, “single”, and other such terminology. Rather, the use of ordinal numbers is to distinguish between the elements. By way of an example, a first element is distinct from a second element, and the first element may encompass more than one element and succeed (or precede) the second element in an ordering of elements.

In the following description, numerous references are cited. All of these references are hereby incorporated by reference in their entirety.

While the disclosure has been described with respect to a limited number of embodiments, those skilled in the art, having benefit of this disclosure, will appreciate that other embodiments may be devised which do not depart from the scope of the disclosure as disclosed herein. Accordingly, the scope of the disclosure should be limited only by the attached claims.

It is to be understood that the singular forms “a,” “an,” and “the” include plural referents unless the context clearly dictates otherwise. Thus, for example, reference to “a horizontal beam” includes reference to one or more of such beams.

Terms like “approximately,” “substantially,” etc., mean that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

As used herein, “omic” refers to a field of study in biology ending in -omics, such as genomics, proteomics, transcriptomics, metabolomics, or other means of molecular analysis used to determine molecular signatures of patient biology and/or tumor. It is envisioned that while techniques discussed in the context of genomics, transcriptomics, and proteomics, may be applied more broadly to encompass other data collections of proteins, small molecules, compounds, and multi-protein interactions.

Although multiple dependent claims are not introduced, it would be apparent to one of ordinary skill in that that the subject matter of the dependent claims of one or more embodiments may be combined with other dependent claims. For example, even though claim 3 does not directly depend from claim 2, even if claim 2 were incorporated into independent claim 1, claim 3 is still able to be combined with independent claim 1 that would now recite the subject matter of dependent claim 2.

In one or more embodiments, thermodynamic measures such as Gibbs Free Energy may be utilized for mapping molecular pathways, also described herein as a molecular subnetwork or PPI subnetwork, for each patient at each stage of cancer progression. This allows selection of molecular targets for treatment with a high confidence that the targets have significant meaning for that patient.

Selected proteins within a PPI network may have greater impact on a network of PPI and may show stronger correlations with a given disease state. It is important to consider, and weight differently in some embodiments, proteins or small molecules such as the products of translation of fusion genes or immune regulators which may show correlation with disease progression. In one or more embodiments, products of gene fusion within a PPI network may be given an energy boost by modifying the associated probability. In some embodiments, immune regulators, such as cytokines, proteoglycans, microvesicles, and the like, may be given an energy boost using a scalar value that is based on a calculated impact on the PPI network.

In general, embodiments of the disclosure describe a linear correlation of Gibbs free energy and cancer patient survival. In one or more embodiments, the Gibbs free energy persistent homology on each cancer PPI network is calculated for each patient. Furthermore, the relevant energetic molecular subnetwork, from which another topological measure called a Betti number or cycle-basis centrality number is used, to select molecular targets for inhibition or activation. Molecular targets may include proteins and peptides, and non-protein products of gene alterations. Because there is a linear correlation with Gibbs free energy, these targets may be selected with confidence. For example, based on the genetic and phenotypic background of an individual, a different proliferative subnetwork may be engaged in tumor growth. In most cancers, more than one genomic and proteomic alteration is usually identified, resulting in a disadvantage situation where the importance of one molecular alteration over another molecular alteration may not be easily determined.

An advantage achieved by one or more embodiments compared to conventional therapy is the high confidence for selecting a molecular alteration, also referred to as the most significant target protein(s), that causes the largest effect on the subnetwork when inhibited or activated. It would be apparent to one of ordinary skill in the art that the molecular alteration that causes the largest effect on the subnetwork would have the largest impact on inhibiting the progression of the cancer.

In general, the phrase “the most significant molecular target(s)” is defined as the protein node(s) in a network or subnetwork that causes the largest change in Betti number or cycle-basis centrality number when removed. In other words, the “most significant” molecular target(s) is the number one or most result-effective molecular target(s) of choice for administering drugs during therapy.

The following examples and description are for explanatory purposes only and not intended to limit the scope of the disclosure.

The homeostasis of cells is maintained by a complex, dynamic network of interacting molecules ranging in size from a few dozen Daltons to hundreds of thousands of Daltons. Any change in concentration of one or more of these molecular species alters the chemical balance, or in terms of thermodynamics, chemical potential. These changes then percolate through the network affecting the chemical potential of other species. The end result is perturbations in the network manifesting as concentration changes, giving rise to changes in the energetic landscape of the cell. In the Third Edition of “Physical Chemistry” published by W.H. Freeman and Company in 1986 and in the “Introduction to Theoretical Organic Chemistry” published by Macmillan Company in 1968, authors P. W. Atkins and A. Liberles, respectively, describe these energetic changes as chemical potential on an energetic landscape.

Gene alterations (mutations, variations in expression, translocations, etc.) invariably alter the chemical potential of one or more proteins and/or other molecular species within a single cell. Yet, two neighboring cancer cells in the same microenvironment may exhibit a different energetic landscape because the chemical potential is different within the two cells. Naturally, when bundles of cells are harvested, for example in a biopsy, and the cells are digested to extract RNA for transcription analysis, the transcriptome is essentially an average of the bundles of cells. Since genes code for proteins, the transcriptome may act as a surrogate for the concentration of the proteins.

To support the conjecture described above, a 2013 publication by Greenbaum et al. on page 117 of volume 4 of Genome Biology titled “Comparing protein abundance and mRNA expression levels on a genomic scale” and a 2009 publication by Maier et al. in pages 3966 to 3973 of volume 583 of the FEBS Letters titled “Correlation of mRNA and protein in complex biological samples,” have described correlations of mRNA with protein concentrations and found Pearson correlation, R, to range from 0.4 to 0.8, in a large number of experiments across five different species. Similarly, as described in a publication titled “Mass-spectrometry-based draft of the human proteome” in pages 582 to 587 of volume 509 of Nature, Wilhelm et al. conducted an extensive study on human tissues using both proteomic and mRNA expression and found roughly an 86% correlation between expression and protein concentration.

Data for several cancers from The Cancer Genome Atlas (TCGA) hosted by the National Institute of Health (www.cancergnome.nih.gov) have been collected. The Cancer Genome Atlas is described by The TGCA Research Network publications in the journal, Nature. A set of data that used the Agilent platform G4502A has also been collected and was pre-collapsed on gene symbols. Further, a total of eleven cancers were collected from the following sources: KIRC (kidney renal clear cell) from a 2013 publication by The TGCA Research Network titled “Comprehensive molecular characterizations of clear cell renal cell carcinoma,” published in pages 43 to 49 of volume 499 of Nature; KIRP (kidney renal papillary cell); LGG (low grade glioma); GBM (glioblastoma multiforme) from a 2008 publication by The TGCA Research Network titled “Comprehensive genetic characterization defines human glioblastoma genes and core pathways,” published in page 1061 of volume 455 of Nature; COAD (colon adenocarcinoma) from a 2012 publication by The TGCA Research Network titled “Comprehensive molecular characterization of human colon and rectal cancer,” published in pages 330 to 337 of volume 487 of Nature; BRCA (breast invasive carcinoma,) from a 2012 publication by The TGCA Research Network titled “Comprehensive molecular portraits of human breast tumors,” published in pages 61 to 70 of volume 490 of Nature; LUAD (lung adenocarcinoma); LUSC (lung squamous cell) from a 2012 publication by The TGCA Research Network titled “Comprehensive genomic characterization of squamous cell lung cancers,” published in pages 519 to 525 of volume 489 of Nature; UCEC (uterine corpus endometrial) from a 2013 publication by The TGCA Research Network titled “Integrated genomic characterization of endometrial carcinoma,” published in pages 67 to 73 of volume 497 of Nature; OV (ovarian serous cystadenocarcinoma) from a 2012 publication by The TGCA Research Network titled “Integrated genomic analysis of ovarian carcinoma,” published in pages 609 to 615 of volume 476 of Nature; READ (rectum adenocarcinoma).

In one or more embodiments, two databases for survival data are used. The first database is the Surveillance Epidemiology and End Results (SEER) National Cancer Institute database, which contains detailed statistical information about the five-year survival rates of patients with cancer. The second database is the National Brain tumor Society database. While these two databases may be used, a single database or multiple other databases could be used that provide the same or equivalent data.

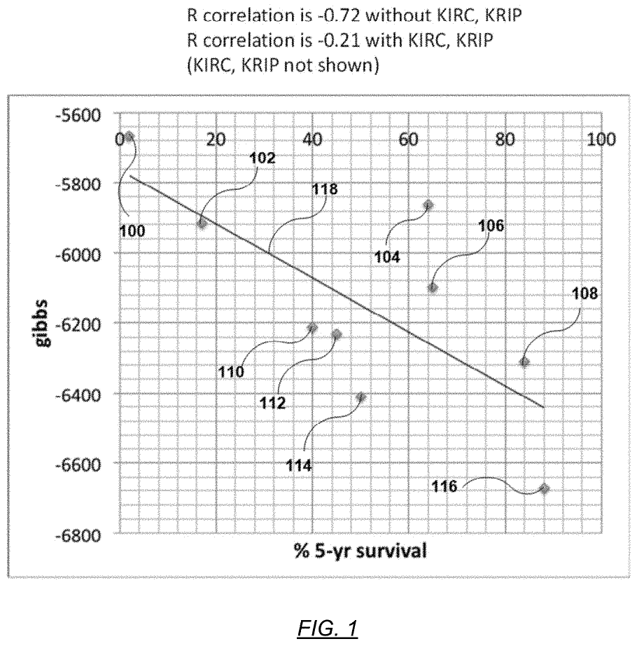

FIG. 1 shows a graph in accordance to one or more embodiments. In one or more embodiments, FIG. 1 shows the application of the TCGA data described above. As seen in FIG. 1, the 5 year survival rate and correlating Gibbs free energy number for the different cancers: glioblastoma multiforme (GMB) (100), lung adenocarcinoma (LUAD) (102), rectum adenocarcinoma (READ) (104), colon adenocarcinoma (COAD) (106), uterine corpus endometrial (UCEC) (108), lung squamous cell (LUSC) (110), ovarian serous cystadenocarcinoma (OV) (112), low grade glioma (LGG) (114), and breast invasive glioma (BRCA) (116) are plotted. The y-axis in FIG. 1 is the Gibbs energy shown in an arbitrary scale and the x-axis represents the probability of 5-year patent survival.

As seen in FIG. 1, a linear correlation (118) exists between overall Gibbs free energy and 5-year survival rate. This result demonstrates that the probability of 5-year patient survival is inversely proportional to the complexity of the signaling network (measured by Gibbs energy) for the types of cancer considered. Other measures of network complexity, such as degree-entropy, number of leaf nodes, and/or cyclomatic number have also been found to inversely correlate with 5-year survival. These results indicate the existence of a correlation between the probability of survival (clinical data) and the complexity of signaling networks (mathematical inference). Furthermore, these results also imply that the inactivation of certain molecular targets (e.g. those that may reduce network complexity) may bring about reduction in cancerous growth and increase in survival.

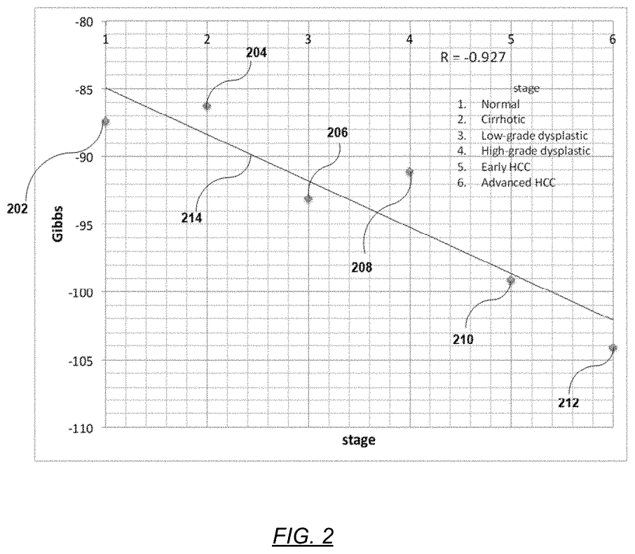

FIG. 2 shows a graph in accordance with one or more embodiments. In one or more embodiments, FIG. 2 is a graph that shows the Gibbs free energy correlation with cancer stage for liver cancer. As shown in FIG. 2, the cancer stages: normal tissue (202), cirrhotic stage (204), low-grade dysplastic (206), high-grade dysplastic (208), early HCC (210), and advanced HCC (212) are assigned to an ordinal number of 1 through 6 and plotted on the x-axis. In FIG. 2, the y-axis is the Gibbs energy on an arbitrary scale In FIG. 2 gene expression data from GSE6764 (publicly available) http://www.ncbi.nlm.nih.gov/geo/query/acc.cgi?acc=GSE6764) was normalized so as to be in the range of [0, 1] and overlaid on a protein-protein interaction network from Biogrid® using Gibbs free energy equations described later in one or more embodiments. In FIG. 2, the Pearson correlation is −0.927, the Spearman correlation of the mean Gibbs free energy for the individual cancer stages is R=−0.99 with a p-value of 0.0001, and the Kendall's tau correlation is 1.000, with a p-value of 0.0016.

As seen in FIG. 2, a linear correlation (214) exists between the Gibbs free energy and the cancer stages when the cancer stages are assigned to an ordinal number. While other protein-protein interaction network measures may have been found to correlate with survival, the finding of a linear correlation between Gibbs energy and cancer stage as shown in FIG. 2 is a new discovery. The results in FIG. 2 provides an additional level of reassurance that changes in network complexity are relevant to cancer progression, because the complexity of each cancer specific protein interaction network can be described by quantifying the energy of the connections within the protein interactions. Therefore, if a decrease in network complexity can be correlated with lower cancer stage, then the identification of nodes (proteins) which produce significant reduction in network complexity may pinpoint the most appropriate therapeutic target.

In one or more embodiments, the Gene Expression Omnibus (GEO) at www.ncbi.nlm.nih.gov is accessed for transcription data relevant to prostate and liver carcinoma. The data for the liver cancer study (hepatocellular carcinoma) was GSE6764, and the prostate study GSE3933 and GSE6099. The GSE3933 and GSE6099, as obtained, were log(2) processed and collapsed to gene IDs. The data was modified to gene cluster text (.gct) file format and processed with GenePattern® at Broad Institute. The expression data for liver cancer, GSE6764, was in an Affymetrix® format (HG_U133_Plus_2 probe set), and also preprocessed to collapse them into gene IDs.

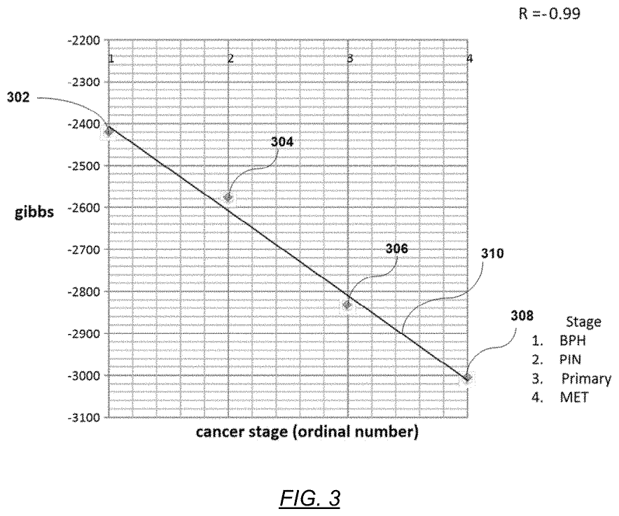

Similarly, FIG. 3 shows a graph in accordance with one or more embodiments. In one or more embodiments, FIG. 3 is a graph showing the Gibbs energy correlating with cancer stage, more specifically, Gibbs free energy vs. cancer stage for prostate cancer. As shown in FIG. 3, the prostate cancer stages: normal benign prostate hypoplasia (BPH) (302), prostatic interepithelial neoplasia (PIN) (304), primary tumor (Primary) (306), and metastatic (MET) (308) are assigned to an ordinal number of 1 through 4 and plotted on the x-axis. In FIG. 3, the y-axis is the Gibbs energy on an arbitrary scale. In one or more embodiments, for the calculation of FIG. 3 gene expression data from GSE3933 and GSE6099 were normalized so as to be in the range of [0, 1] and overlaid on Biogrid® protein-protein interaction network using Gibbs free energy equations described later in one or more embodiments. In FIG. 3, the Spearman R correlation is −1.000 with p-value.

As seen in FIG. 3, a linear correlation (310) exists between the Gibbs free energy and the cancer stages when the cancer stages are assigned to an ordinal number. As described above in FIG. 2, the identification of protein hubs that most contribute to network complexity (most energetic nodes) is likely to pinpoint putative molecular targets for therapy. Carefully choosing a minimum set of molecular targets to be inhibited, according to the subnetwork energy, may result in a X % decrease in the calculated network complexity (measured by Gibbs energy), and may double the predicted rate of 5-year survival or reduce cancer stage.

It would be apparent to one of ordinary skill in the art that given that the data for these calculations come from such diverse sources it is highly suggestive that the correlations are good. This suggests exploiting the Gibbs energy concept for target selection.

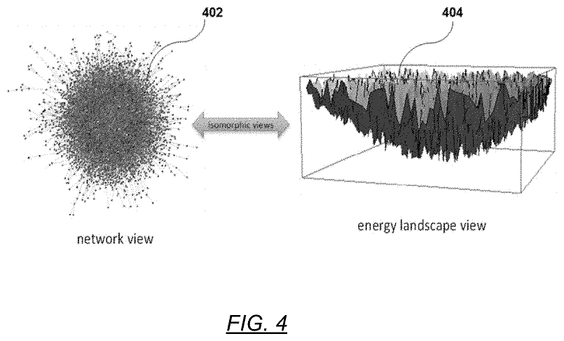

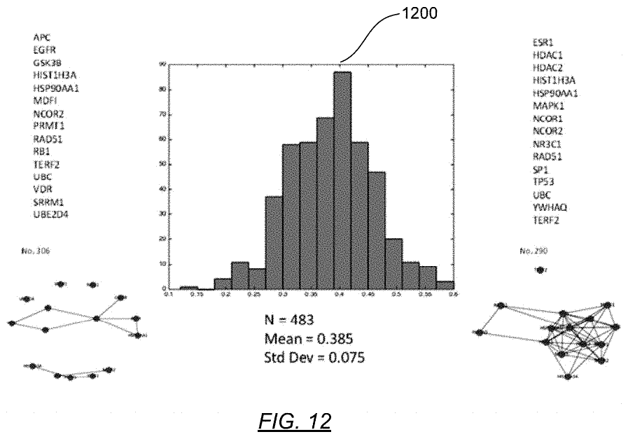

FIG. 4 shows diagrams in accordance with one or more embodiments. In one or more embodiments, once a Gibbs free energy is assigned to each node in a PPI network, the PPI network may then be viewed as a rugged landscape (402) within, for example, a graphical user interface (GUI) (401) as depicted in FIG. 4. In one or more embodiments of the disclosure, the GUI and one or more display devices for viewing the GUI is shown and described in relation to FIG. 12A. Returning to FIG. 4, the network with real numbers attached to each node is isomorphic to an energy landscape (404), which in one or more embodiments is displayed within the GUI (401). A topological “filtration” technique may be applied to the energy landscape (404) to extract a “persistent homology.”

In one or more embodiments, the human PPI network (Homo sapiens, 3.3.99, March, 2013) from BioGrid (www.thebiogrid.org), which contains 9561 nodes and 43,086 edges, was used. The entire human PPI was loaded into version 2.8.1 of Cytoscape. In a publication by Shannon et al. titled “Cytoscape: A softward environment for integrated models of bimolecular interaction networks,” published in 2013 in pages 2498 to 2504 of volume 13 issue 11 of Genome Research, Shannon et al. describes the general application and use of the Cytoscape software. The list of genes obtained from TCGA (full-length expression set was 17,814 genes) for a specific cancer was “selected” using the Cytoscape functions, the “inverse selection” of Cytoscape function applied, and the nodes and genes edges were removed. The resulting network, which now included only those genes found in both Biogrid and TCGA, consisted of 7951 nodes and 36,509 edges. This Cytoscape network was unloaded as an adjacency list for processing by custom Python code using version 2.6.4 of Python with appropriate NetworkX functions.

In one or more embodiments the RNA (e.g., mRNA, rRNA, tRNA, and other non-coding RNA) transcriptome value as a surrogate for protein concentration may be “overlaid” on a PPI network, such as the human PPI at Biogrid (www.biogrid.org) shown as the rugged landscape (402) in FIG. 4. Once the RNA transcriptome value has been overlaid, the log(2) transformed transcription data is first rescaled to be in the range [0, 1]. In one or more embodiments, the most highly, positively expressed value will be set to 1.0 and the most negatively, down-regulated value will be set to 0.

It would be apparent to one of ordinary skill in the art that this is comparable to stating that the most strongly up-regulated gene produces a protein of very great concentration, relative to the most strongly down-regulated gene that will result in the lowest protein concentration.

In one or more embodiments, the corresponding rescaled transcriptome data is assigned to each protein in the PPI network. The following equation is then used to compute the Gibbs free energy for that protein:

$${G}_{i} = {c}_{i} ln \frac{{c}_{i}}{{\sum }_{j = i} {c}_{j}} \qquad Eq . [ 1 ]$$

In one or more embodiments, it is assumed that the protein of interest is i with concentration, ci. This concentration is the rescaled transcription data for that gene. In the denominator of the argument to the natural logarithm the summation is taken over concentrations (rescaled) for all the neighbors to the protein of interest, i. This is essentially the Gibbs free energy, Gi, for that protein in the PPI network.

In one or more embodiments, the overall Gibbs free energy of the PPI network may be obtained using the equation of:

$$qG = {\sum }_{i} {G}_{i} \qquad Eq . [ 2 ]$$

In one or more embodiments, Equation [2] represents the Gibbs free energy for a patient. In one or more embodiments, Equation [2] may also represent the different cancer stages for patients, depending on when the biopsy was taken.

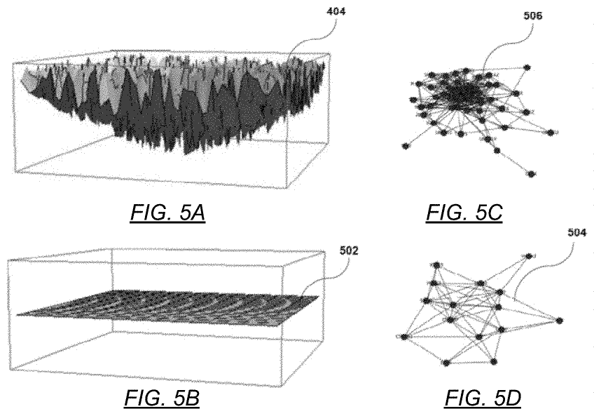

FIGS. 5A, 5B, 5C, and 5D show diagrams in accordance with one or more embodiments. In one or more embodiments, shown in FIG. 5A, an energy landscape (404) is shown. In one or more embodiments, shown in FIG. 5B, a topological filtration (502), also referred to as a filtration threshold, may be moved up from far below the lowest minima on an energy landscape (404). As the filtration threshold is moved up further, small connected subnetworks (504) as shown in FIG. 5D, and later larger connected subnetworks (506) as shown in FIG. 5C are revealed. These subnetworks are known as persistent homology.

As shown in FIGS. 5C and 5D, it would be apparent to one of ordinary skill in the art that as the filtration threshold is increased, the complexity of the subnetwork is also increased.

In one or more embodiments, if the normalized or rescaled, expression data were assigned as real numbers a persistent homology cannot be obtained when the topological filtration is applied. The nodes will be disconnected until a threshold of several hundred. In contrast, by using the normalized or rescaled, expression data, a user set threshold as low as 1 and as high as 20,000 gives a smooth change in network measure on the subnetworks.

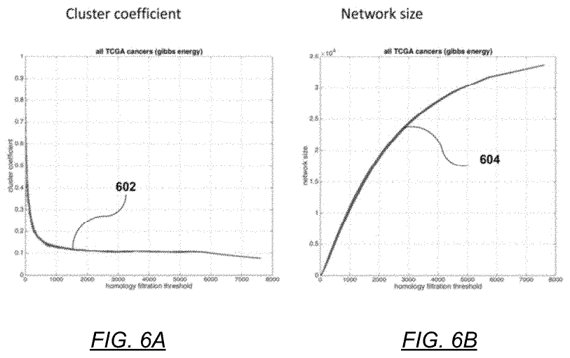

FIGS. 6A and 6B shows a graph in accordance with one or more embodiments. In one or more embodiments, the graph in FIG. 6A shows the cluster coefficient and the graph in FIG. 6B shows the cluster size of the persistent homology subnetworks as a functions of the filtration threshold. As shown in the first curve (602) in FIG. 6A and the second curve (604) in FIG. 6B, no apparent kinks are shown that would represent a phase transition as the filtration threshold is increased from 1 to 7000.

In one or more embodiments, to demonstrate how the subnetworks are used for targeting and treatment of individual patients, the TCGA glioblastoma multiforme (GBM) data is used as an example.

In one or more embodiments, FIG. 12 shows a histogram (1200) for a network metric known as closeness centrality, which measures the mean distance from a node in the network to all other nodes, on 483 GBM patients. The Gibbs energy persistent homology for the individual patient was first computed, and the closeness centrality for subnetworks at a filtration threshold of 15 was then computed. In one or more embodiments, the histogram in FIG. 12 shows the full range of closeness centrality and thus the differences in subnetworks for each patient.

As shown in FIG. 12, the graph in the center presents the distribution of closeness-centrality (the x-axis) vs. the number of subnetworks at a filtration threshold of 15 (the y-axis). On the left of the graph, a list of genes with the respective subnetwork is provided. This subnetwork represents an example of the least connected network (e.g. one that has the lowest closeness-centrality of the population of graphs). On the right of the graph, another list of genes with the respective subnetwork is provided. In contrast to the list of genes on the left, the list of genes provided on the right contains the most connected network (e.g., the highest closeness-centrality of the population of graphs).

In one or more embodiments, the distribution study as shown in FIG. 12 refers to a population of patients and therefore identifies frequency of specific homology subnetworks within a population of patients with specific type of cancer and guide drug treatment for the majority of patients vs rare molecular subtypes.

In one or more embodiments, the subnetworks may be used to compute drug targets. First, the Gibbs energy of the subnetwork is demonstrated as significant, in relation to survival of GBM patients. In one or more embodiments, a Cox proportional hazards (Cox PH) model is used to show this significance.

The Cox proportional hazards were described by Cox in a 1972 publication titled “Regression Models and Life Tables” in pages 187 to 220 in series B, volume 34, No. 2 of the Journal of Royal Statistical Society.

In a research paper titled “Molecular signaling network complexity is correlated with cancer patient survivability” published in 2012 in volume 109 issue 23 of the Proceedings of the National Academy of Sciences, Breitkreutz et al. shows that the model was constructed from several statistical and thermodynamic measures on the Gibbs subnetwork at threshold of 32. The statistical measures included: number of edges, transitivity, and clique.

Furthermore, a topological measure known as the Betti number is used. The Betti number is described by Benzekry et al. in a publication titled “Design Principles for Cancer Therapy guided by changes in complexity of Protein-Protein Interaction Networks.” The Betti number calculates the number of rings of four or more nodes in the PPI network, in this case the Gibbs homology subnetworks. The cycle-basis centrality is an alternate calculation for the first Betti number of a topological space.

These seven parameters (i.e. number of edges, transitivity, clique, degree-entropy, Betti number, cycle-basis centrality number, Gibbs energy of the subnetwork) are fitted into the Cox PH model. The Chi Square probability for the overall model is 0.0426 and the most important parameter is the Gibbs energy of the subnetwork with a Chi Square fitting probability of 0.0026. Furthermore, fitting only to days-to-death with Gibbs-subnetwork energy in log-logistic model, a Chi square of <0.0001 is obtained.

In one or more embodiments, the Betti number or cycle-basis centrality number and the Gibbs energy for this subnetwork is calculated. It would be apparent that since Betti number and Gibbs free energy correlates linearly with survival for different cancers, it is possible to inhibit a protein at different stages of the cancer that gives the largest drop in Betti number with high confidence that the complexity of the subnetwork has been reduced.

In one or more embodiments, whether or not the complexity has been reduced may be double checked to see if the Gibbs free energy has increase. In one or more embodiments, this is done on a patient-to-patient basis. It would be apparent to one of ordinary skill in the art that the method of one or more embodiments, referred to as the Gibbs-Betti method, may generate an energetic subnetwork for each patient no matter the cancer stage. Furthermore, the Gibbs-Betti method of one or more embodiments may be used to identify a specific drug target for each patient.

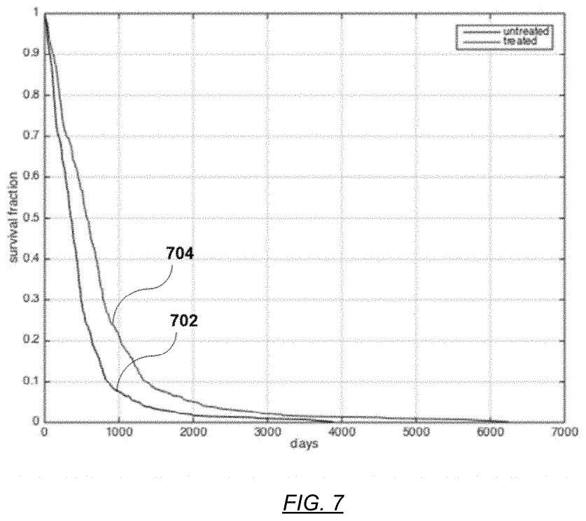

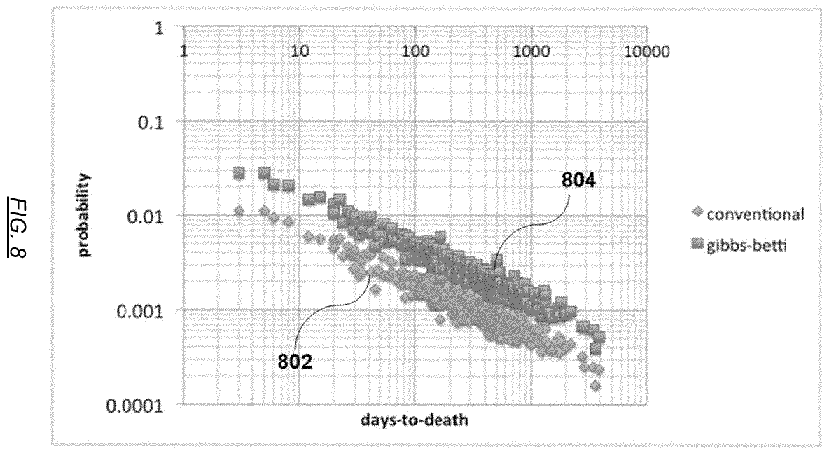

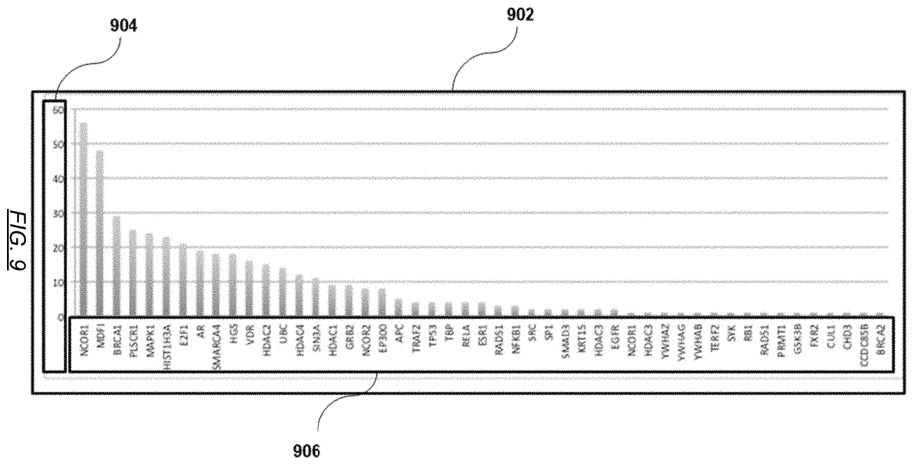

FIG. 7 shows a graph according to one or more embodiments. In one or more embodiments, FIG. 7 shows a hazard model: a fit of days-to-death with Gibbs energy for the homology subnetworks at threshold 32 for glioblastoma (using the same TCGA data), which is also referred to as a log-logistic fit. The lowest curve (702) represents the untreated patients, and the upper curve (704) is a simulation of patients treated with targeted agents that inhibit the proteins identified using the Gibbs-homology (threshold 32) and Betti number or cycle-basis centrality number method as described above in one or more embodiments. The x-axis represents number of days to death (from TCGA), and the y-axis is survival fraction (or probability of survival).

From the results shown in the graph of one or more embodiments in FIG. 7, it would be apparent to one of ordinary skill in the art that the patients treated with the Gibbs-Betti method of one or more embodiments survived longer than the conventionally treated patients.

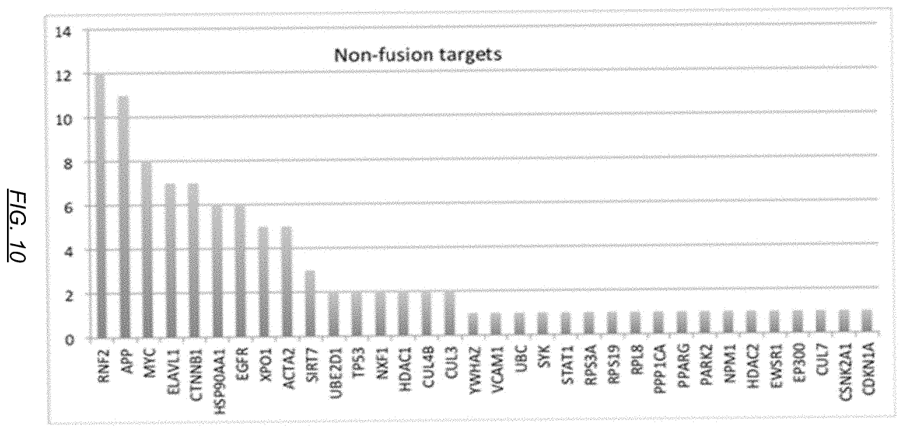

FIG. 8 shows a graph according to one or more embodiments. As seen in FIG. 8, the log-logistic for glioblastoma patients as shown in FIG. 7 treated with conventional therapy (802) and glioblastoma patients with a simulated treatment based on the Gibbs-Betti method (804) of one or more embodiments are compared. The overall improvement of the Gibbs-Betti method (804) of one or more embodiments compared to the results for conventional therapy (802) is estimated at 134%.

FIG. 9 shows a graph in accordance with one or more embodiments. FIG. 9 shows a Pareto chart (902) of the best targets for individual patients (904) with glioblastoma carcinoma. The chart shows that the best molecular target among the plurality of molecular targets (906) was NCOR1 for 56 patients. The chart also shows that MDF1 was the best target for 48 patients.

While some embodiments may set all proteins in a PPI network on the same level, it is also envisioned that certain protein constructs (such as proteins translated from gene fusions) should be regarded as more important and placed on a tier that is weighed more heavily in statistical calculations for enhanced analysis. In some contexts, a number of identified proteins may be associated with certain disease states more frequently than other proteins in a PPI. For example, proteins implicated in cancer states, proteins originating from gene fusions, inflammation, or immune disorders may be correspondingly be given greater weight or importance in statistical calculations in accordance with the present disclosure.

Cancer is a disease of multiple alterations, with single mutations infrequently resulting in a cancer. One hallmark of cancer is genome instability and mutation in which multiple alterations negatively impact chromosome structure and function, resulting in a “shattering” of chromosomes. Dysfunctional chromosomes may possess multiple gene copies, copies of entire chromosomal regions, or diminished gene copy numbers or chromosomal deletions relative to a healthy chromosome. One of the possible consequences of “chromosome shattering” is gene fusion, often across different chromosomes. Genes code for mRNA, and often those mRNAs code for proteins. If two genes fuse as a result of chromosome rearrangement, the resulting new gene may code for a protein fusion product. Gene fusions may be indicators of the presence of key molecular systems for the survival of some types of cancer. Here, a molecular system refers to the proteins expressed from the fusion gene that forms larger proteins and complexes than the proteins generated from the original gene constructs.

Fusion proteins generated from gene fusions may be composed of, for example, a large-length piece of one protein and a medium-length piece of another protein. Fusion proteins may travel unique folding pathways to generate complex 3-dimensional shapes driven by entropy, which may have the net result that drugs targeting one of the constituent proteins (usually by protein inhibition), may target fusion proteins as well. The molecular targeting works on this fusion protein because some regions of the folded structure resemble the native folded protein.

Clinicians have identified the common protein fusion products (which proteins are fused to which other) and from meta-analysis of many cancers they have also identified the probability of these fusions (Yoshihara, Wang, Torress-Garcia, Zheng, Vegesna, Kim, Verhaak, “The landscape and therapeutic relevance of cancer-associated transcript fusions” Oncogene (2015), 34, 4845-4854 . . . see FIG. 1, page 4847, and Supplemental Tables). We may exploit this probability information in our analysis of Gibbs energy and thus Gibbs-homology for enhanced “target” identification.

To exploit these probabilities an energy level diagram, or synonymously energy distribution may be employed in methods of constructing PPI networks in accordance with the present disclosure. In one or more embodiments, a PPI network may have an initial distribution or “ground state” energy level, with a number of levels that are assigned as “higher energy.”

In embodiments discussed above, an algorithm may put all proteins on the same level in the PPI network. None are said to be more important than any other. The gene expression data (e.g., mRNA transcription or RNAseq) provides a measure of importance. Higher-expression genes as modulated by their neighbors expression data and interconnectivity may result in greater chemical potential and thus higher Gibbs free energy.

In one or more embodiments, methods in accordance with the present disclosure may put products of gene fusion as probabilities on higher energy levels in the PPI network. Using a modification of the concept of growing networks on a Fermi energy level diagram, from Bianconi, Barabasi, “Bose-Einstein Condensation in Complex Networks”, Physical Rev. Lett. 86, (24), 5632-5635, Jun. 11, 2001, methods in accordance with the present disclosure may incorporate a Fermi distribution energy level. Bianconi and Barabasi discuss that the probability of connecting a new node to an existing node i, from one level to the next is given by Eq. [3], where ηᵢ is the probability fitness parameter, and kᵢ is the energy level for node i.

$${\prod }_{i} = \frac{{η}_{i} {k}_{i}}{{\sum }_{I} {η}_{I} {k}_{I}} \qquad Eq [ 3 ]$$

If the number of nodes at a given level is high the denominator may be very large, thus driving the probability up. Nodes may then be grown to a network by connecting nodes from one level to the next.

In one or more embodiments, network construction may include a number of levels representing different probability levels, and at each level the nodes may or may not be connected to each other. The “ground state” is defined as the first level. In some embodiments, the ground state represents the conventional BioGrid PPI (e.g., 20,000+ nodes, ˜220,000+ edges), while the next energy level above ground state may be assigned a designation as level 1.1, the next may be assigned 1.2, etc. Higher energy proteins such as products of gene fusion and other disease state associated proteins may then be assigned as higher probability nodes; level 2.0, for example. In some embodiments, node labeling may be used to indicate different connectivity between each node level. For example, nodes at level 1.5 that have the same node labels as nodes at the ground state and may represent two networks with same labels but different connectivity—different networks. Networks at the levels above the ground state are probability fusion networks but have the same node labels as the much larger network at the ground state, 1.0. Thus, if the union of all networks is constructed from the ground state, 1.0, to highest state, 2.0, a network map of conventional BioGrid PPI that now includes connections to probability fusion genes may be generated. This will effectively introduce new connections between existing nodes in the PPI that assigns greater import to potentially relevant clinical targets such as products of gene fusion, immunological proteins, and the like.

Now to exploit this for chemical potential purposes, or Gibbs free energy, a subset of nodes may be assigned higher energy levels. In one or more embodiments, nodes may be associated with a scalar number, a probability value, representing the energy level to each node in the network formed by the union of all networks from all levels. In cases where the expression data is supplemented by genomic tests that empirically validate one or more fusions for a given patient, we set the probability for those fusions to 1. To now compute the Gibbs free energy for a node, i, in the PPI Eq [1] is modified to give Eq [4], where E_{α}⁽ⁱ⁾ represents energy level α, for a given node, and the symbol (i) reminds us that are looking at node i.

$${G}_{i} = ( {c}_{i} + {E}_{α}^{( i )} ) ln ( \frac{{c}_{i} + {E}_{α}^{( i )}}{{\sum }_{j = i} ( {c}_{j} + {E}_{α}^{( j )} )} ) \qquad Eq . [ 4 ]$$

As an example, if the node is at energy level, 1.1, this represents the sum of the ground state energy, 1.0 and the probability, p=0.1. As indicated by, E_{α}^{(j)}, the energy level for each node needs to be considered in the summation. In summary, every normalized expression value, cⱼ, is boosted by summing with the probability of fusion. Eq. [4] thus gives the Gibbs free energy for each node. The typical Gibbs homology, and Betti number or cycle-basis centrality number may now be calculated as described above.

In the next example, “Betti targets”—the proteins selected for inhibition—are compared between the fusion and non-fusion approaches. For this demonstration, READ (rectal adenocarcinoma) data was obtained from publicly available TCGA data (https://cancergenome.nih.gov/) for 72 patients. A lookup table was built from the fusion probabilities per gene (data from Yoshihara, 2015). So the table consisted of gene ID and probability of its being involved in a gene fusion. Gene fusions are considered actual proteins that, while being covalently attached, “interact” from a PPI network perspective. Networks having 10 levels in a Fermi-like distribution were then constructed, numbering from 1, ground state, to the highest probability state, 2.0.

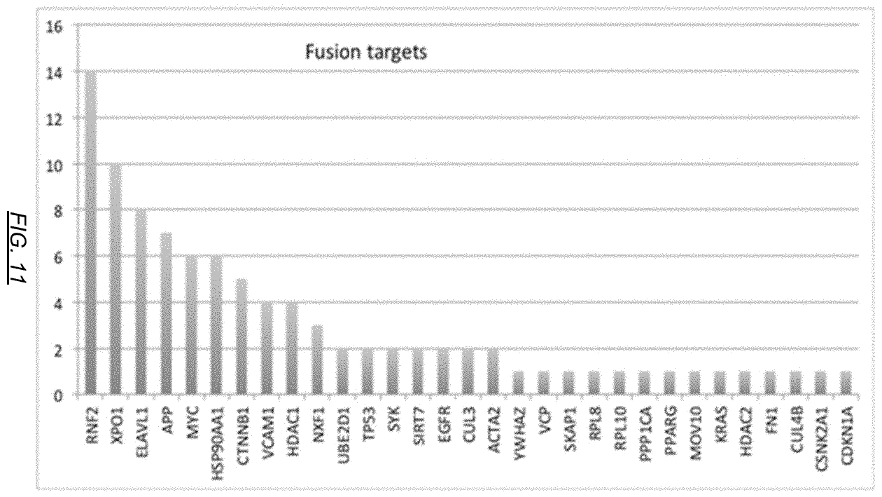

Finding the union of all networks involves merging the networks at differing levels into a single network. Associated with each node now was the modified expression, or concentration, and the energy level (e.g. ground state→1.0; highest state→2.0), exactly as indicated in the above Eq. [4]. The Gibbs free energy for each node, the Gibbs homology, and best target based on Betti number or cycle-basis centrality number were then calculated for the system. Carrying out these calculations on the TCGA READ data we get the results shown in FIGS. 10 and 11, which show Pareto charts of targets computed from non-fusion networks and networks that account for known fusion genes, respectively.

It is noted that were 72 patients of data from this study and there were some cases of dual equivalent targets, such that the total number of targets is greater than 72. Comparing the two Pareto charts, most of the “high occurring” targets are the same genes but they differ only in a few patient-occurrences. Also interesting are the “low occurrence” targets, which differ widely between the two Pareto charts in FIGS. 10 and 11. From a practical perspective, this suggests actual clinical treatment differences between the two batches of patients, depending on the methodology used.

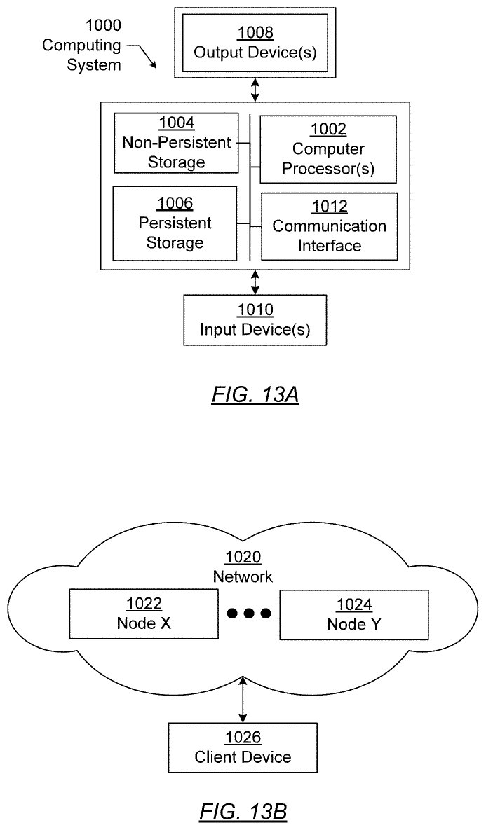

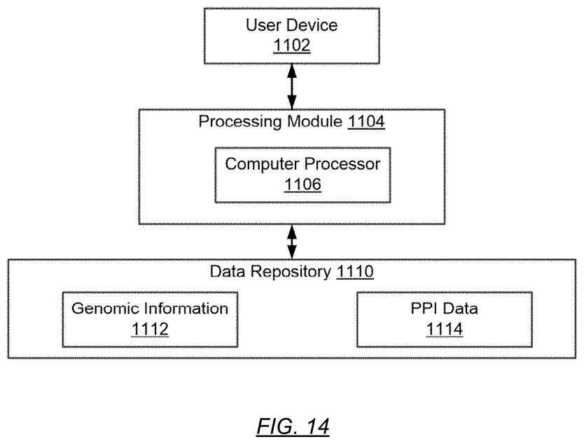

FIGS. 13A and 13B show a computing system in accordance with one or more embodiments of the technology. Embodiments of the disclosure may be implemented on a computing system. Any combination of mobile, desktop, server, router, switch, embedded device, or other types of hardware may be used. For example, as shown in FIG. 13A, the computing system (1000) may include one or more computer processors (1002), non-persistent storage (1004) (e.g., volatile memory, such as random access memory (RAM), cache memory), persistent storage (1006) (e.g., a hard disk, an optical drive such as a compact disk (CD) drive or digital versatile disk (DVD) drive, a flash memory, etc.), a communication interface (1012) (e.g., Bluetooth interface, infrared interface, network interface, optical interface, etc.), and numerous other elements and functionalities.

The computer processor(s) (1002) may be an integrated circuit for processing instructions. For example, the computer processor(s) may be one or more cores or micro-cores of a processor. The computing system (1000) may also include one or more input devices (1010), such as a touchscreen, keyboard, mouse, microphone, touchpad, electronic pen, or any other type of input device.

The communication interface (1012) may include an integrated circuit for connecting the computing system (1000) to a network (not shown) (e.g., a local area network (LAN), a wide area network (WAN) such as the Internet, mobile network, or any other type of network) and/or to another device, such as another computing device.

Further, the computing system (1000) may include one or more output devices (1008), such as a screen (e.g., a liquid crystal display (LCD), a plasma display, touchscreen, cathode ray tube (CRT) monitor, projector, or other display device), a printer, external storage, or any other output device. One or more of the output devices may be the same or different from the input device(s). The input and output device(s) may be locally or remotely connected to the computer processor(s) (1002), non-persistent storage (1004), and persistent storage (1006). Many different types of computing systems exist, and the aforementioned input and output device(s) may take other forms.

Software instructions in the form of computer readable program code to perform embodiments of the disclosure may be stored, in whole or in part, temporarily or permanently, on a non-transitory computer readable medium such as a CD, DVD, storage device, a diskette, a tape, flash memory, physical memory, or any other computer readable storage medium. Specifically, the software instructions may correspond to computer readable program code that, when executed by a processor(s), is configured to perform one or more embodiments of the disclosure.

The computing system (1000) in FIG. 13A may be connected to or be a part of a network. For example, as shown in FIG. 13B, the network (1020) may include multiple nodes (e.g., node X (1022), node Y (1024)). Each node may correspond to a computing system, such as the computing system shown in FIG. 13A, or a group of nodes combined may correspond to the computing system shown in FIG. 13A. By way of an example, embodiments of the disclosure may be implemented on a node of a distributed system that is connected to other nodes. By way of another example, embodiments of the disclosure may be implemented on a distributed computing system having multiple nodes, where each portion of the disclosure may be located on a different node within the distributed computing system. Further, one or more elements of the aforementioned computing system (1000) may be located at a remote location and connected to the other elements over a network.

Although not shown in FIG. 13B, the node may correspond to a blade in a server chassis that is connected to other nodes via a backplane. By way of another example, the node may correspond to a server in a data center. By way of another example, the node may correspond to a computer processor or micro-core of a computer processor with shared memory and/or resources.

The nodes (e.g., node X (1022), node Y (1024)) in the network (1020) may be configured to provide services for a client device (1026). For example, the nodes may be part of a cloud computing system. The nodes may include functionality to receive requests from the client device (1026) and transmit responses to the client device (1026). The client device (1026) may be a computing system, such as the computing system shown in FIG. 13A. Further, the client device (1026) may include and/or perform all or a portion of one or more embodiments of the disclosure.

The computing system or group of computing systems described in FIGS. 13A and 13B may include functionality to perform a variety of operations disclosed herein. For example, the computing system(s) may perform communication between processes on the same or different system. A variety of mechanisms, employing some form of active or passive communication, may facilitate the exchange of data between processes on the same device. Examples representative of these inter-process communications include, but are not limited to, the implementation of a file, a signal, a socket, a message queue, a pipeline, a semaphore, shared memory, message passing, and a memory-mapped file. Further details pertaining to a couple of these non-limiting examples are provided below.

Based on the client-server networking model, sockets may serve as interfaces or communication channel end-points enabling bidirectional data transfer between processes on the same device. Foremost, following the client-server networking model, a server process (e.g., a process that provides data) may create a first socket object. Next, the server process binds the first socket object, thereby associating the first socket object with a unique name and/or address. After creating and binding the first socket object, the server process then waits and listens for incoming connection requests from one or more client processes (e.g., processes that seek data). At this point, when a client process wishes to obtain data from a server process, the client process starts by creating a second socket object. The client process then proceeds to generate a connection request that includes at least the second socket object and the unique name and/or address associated with the first socket object. The client process then transmits the connection request to the server process. Depending on availability, the server process may accept the connection request, establishing a communication channel with the client process, or the server process, busy in handling other operations, may queue the connection request in a buffer until server process is ready. An established connection informs the client process that communications may commence. In response, the client process may generate a data request specifying the data that the client process wishes to obtain. The data request is subsequently transmitted to the server process. Upon receiving the data request, the server process analyzes the request and gathers the requested data. Finally, the server process then generates a reply including at least the requested data and transmits the reply to the client process. The data may be transferred, more commonly, as datagrams or a stream of characters (e.g., bytes).

Shared memory refers to the allocation of virtual memory space in order to substantiate a mechanism for which data may be communicated and/or accessed by multiple processes. In implementing shared memory, an initializing process first creates a shareable segment in persistent or non-persistent storage. Post creation, the initializing process then mounts the shareable segment, subsequently mapping the shareable segment into the address space associated with the initializing process. Following the mounting, the initializing process proceeds to identify and grant access permission to one or more authorized processes that may also write and read data to and from the shareable segment. Changes made to the data in the shareable segment by one process may immediately affect other processes, which are also linked to the shareable segment. Further, when one of the authorized processes accesses the shareable segment, the shareable segment maps to the address space of that authorized process. Often, only one authorized process may mount the shareable segment, other than the initializing process, at any given time.

Other techniques may be used to share data, such as the various data described in the present application, between processes without departing from the scope of the disclosure. The processes may be part of the same or different application and may execute on the same or different computing system.

Rather than or in addition to sharing data between processes, the computing system performing one or more embodiments of the disclosure may include functionality to receive data from a user. For example, in one or more embodiments, a user may submit data via a GUI on the user device. Data may be submitted via the graphical user interface by a user selecting one or more graphical user interface widgets or inserting text and other data into graphical user interface widgets using a touchpad, a keyboard, a mouse, or any other input device. In response to selecting a particular item, information regarding the particular item may be obtained from persistent or non-persistent storage by the computer processor. Upon selection of the item by the user, the contents of the obtained data regarding the particular item may be displayed on the user device in response to the user's selection.

By way of another example, a request to obtain data regarding the particular item may be sent to a server operatively connected to the user device through a network. For example, the user may select a uniform resource locator (URL) link within a web client of the user device, thereby initiating a Hypertext Transfer Protocol (HTTP) or other protocol request being sent to the network host associated with the URL. In response to the request, the server may extract the data regarding the particular selected item and send the data to the device that initiated the request. Once the user device has received the data regarding the particular item, the contents of the received data regarding the particular item may be displayed on the user device in response to the user's selection. Further to the above example, the data received from the server after selecting the URL link may provide a web page in Hyper Text Markup Language (HTML) that may be rendered by the web client and displayed on the user device.

Once data is obtained, such as by using techniques described above or from storage, the computing system, in performing one or more embodiments of the disclosure, may extract one or more data items from the obtained data. For example, the extraction may be performed as follows by the computing system in FIG. 13A. First, the organizing pattern (e.g., grammar, schema, layout) of the data is determined, which may be based on one or more of the following: position (e.g., bit or column position, Nth token in a data stream, etc.), attribute (where the attribute is associated with one or more values), or a hierarchical/tree structure (consisting of layers of nodes at different levels of detail—such as in nested packet headers or nested document sections). Then, the raw, unprocessed stream of data symbols is parsed, in the context of the organizing pattern, into a stream (or layered structure) of tokens (where each token may have an associated token “type”).

Next, extraction criteria are used to extract one or more data items from the token stream or structure, where the extraction criteria are processed according to the organizing pattern to extract one or more tokens (or nodes from a layered structure). For position-based data, the token(s) at the position(s) identified by the extraction criteria are extracted. For attribute/value-based data, the token(s) and/or node(s) associated with the attribute(s) satisfying the extraction criteria are extracted. For hierarchical/layered data, the token(s) associated with the node(s) matching the extraction criteria are extracted. The extraction criteria may be as simple as an identifier string or may be a query presented to a structured data repository (where the data repository may be organized according to a database schema or data format, such as XML).

The extracted data may be used for further processing by the computing system. For example, the computing system of FIG. 13A, while performing one or more embodiments of the disclosure, may perform data comparison. Data comparison may be used to compare two or more data values (e.g., A, B). For example, one or more embodiments may determine whether A>B, A=B, A !=B, A<B, etc. The comparison may be performed by submitting A, B, and an opcode specifying an operation related to the comparison into an arithmetic logic unit (ALU) (i.e., circuitry that performs arithmetic and/or bitwise logical operations on the two data values). The ALU outputs the numerical result of the operation and/or one or more status flags related to the numerical result. For example, the status flags may indicate whether the numerical result is a positive number, a negative number, zero, etc. By selecting the proper opcode and then reading the numerical results and/or status flags, the comparison may be executed. For example, in order to determine if A>B, B may be subtracted from A (i.e., A−B), and the status flags may be read to determine if the result is positive (i.e., if A>B, then A−B>0). In one or more embodiments, B may be considered a threshold, and A is deemed to satisfy the threshold if A=B or if A>B, as determined using the ALU. In one or more embodiments of the disclosure, A and B may be vectors, and comparing A with B requires comparing the first element of vector A with the first element of vector B, the second element of vector A with the second element of vector B, etc. In one or more embodiments, if A and B are strings, the binary values of the strings may be compared.

The computing system in FIG. 13A may implement and/or be connected to a data repository. For example, one type of data repository is a database. A database is a collection of information configured for ease of data retrieval, modification, re-organization, and deletion. Database Management System (DBMS) is a software application that provides an interface for users to define, create, query, update, or administer databases.

The user, or software application, may submit a statement or query into the DBMS. Then the DBMS interprets the statement. The statement may be a select statement to request information, update statement, create statement, delete statement, etc. Moreover, the statement may include parameters that specify data, or data container (database, table, record, column, view, etc.), identifier(s), conditions (comparison operators), functions (e.g. join, full join, count, average, etc.), sort (e.g. ascending, descending), or others. The DBMS may execute the statement. For example, the DBMS may access a memory buffer, a reference or index a file for read, write, deletion, or any combination thereof, for responding to the statement. The DBMS may load the data from persistent or non-persistent storage and perform computations to respond to the query. The DBMS may return the result(s) to the user or software application.

The computing system of FIG. 13A may include functionality to present raw and/or processed data, such as results of comparisons and other processing. For example, presenting data may be accomplished through various presenting methods. Specifically, data may be presented through a user interface provided by a computing device. The user interface may include a GUI that displays information on a display device, such as a computer monitor or a touchscreen on a handheld computer device. The GUI may include various GUI widgets that organize what data is shown as well as how data is presented to a user. Furthermore, the GUI may present data directly to the user, e.g., data presented as actual data values through text, or rendered by the computing device into a visual representation of the data, such as through visualizing a data model.

For example, a GUI may first obtain a notification from a software application requesting that a particular data object be presented within the GUI. Next, the GUI may determine a data object type associated with the particular data object, e.g., by obtaining data from a data attribute within the data object that identifies the data object type. Then, the GUI may determine any rules designated for displaying that data object type, e.g., rules specified by a software framework for a data object class or according to any local parameters defined by the GUI for presenting that data object type. Finally, the GUI may obtain data values from the particular data object and render a visual representation of the data values within a display device according to the designated rules for that data object type.

Data may also be presented through various audio methods. In particular, data may be rendered into an audio format and presented as sound through one or more speakers operably connected to a computing device.

Data may also be presented to a user through haptic methods. For example, haptic methods may include vibrations or other physical signals generated by the computing system. For example, data may be presented to a user using a vibration generated by a handheld computer device with a predefined duration and intensity of the vibration to communicate the data.

The above description of functions presents only a few examples of functions performed by the computing system of FIG. 13A and the nodes and/or client device in FIG. 13B. Other functions may be performed using one or more embodiments of the disclosure.

FIG. 14 shows a schematic diagram of a system in accordance with one or more embodiments. The system for selecting a molecular target for therapeutic application includes (i) a processing module (1104) including a computer processor (1106) configured to execute instructions configured to: access genomic information (transcription/gene expression analysis, rare transcript, splice variant or fusion transcript on any of the present or future analytic platforms) associated with a patient, access PPI data from one or more reference human (academic, public or private) PPI networks, compute, using the genomic information and the PPI data, a thermodynamic or mathematical measure, and determine, from the thermodynamic or mathematical measure, a molecular target within the PPI data; and (ii) a user device (1102) configured to present the molecular target to a user. The system may further include a data repository (1110) configured to store the genomic information (1112) and the PPI data (1114), in addition to patient data and meta data.

FIGS. 15, 16, 17A, 17B, 18A, 18B, and 18C show flowcharts of methods in accordance with one or more embodiments. In one or more embodiments, the method as shown in FIGs. FIGS. 15, 16, 17A, 17B, 18A, 18B, and 18C are computer-implemented methods. Each step shown in FIGs. FIGS. 15, 16, 17A, 17B, 18A, 18B, and 18C is described below.

While the various steps in these flowcharts are presented and described sequentially, one of ordinary skill will appreciate that some or all of the steps may be executed in different orders, may be combined or omitted, and some or all of the steps may be executed in parallel. Furthermore, the steps may be performed actively or passively. For example, some steps may be performed using polling or be interrupt driven in accordance with one or more embodiments of the invention. By way of an example, determination steps may not require a processor to process an instruction unless an interrupt is received to signify that condition exists in accordance with one or more embodiments of the invention. As another example, determination steps may be performed by performing a test, such as checking a data value to test whether the value is consistent with the tested condition in accordance with one or more embodiments of the invention.

Turning to FIG. 15, a method for selecting a molecular target for therapeutic applications, in accordance with one or more embodiments, is shown. FIG. 15 is intended to provide an overview, with the subsequent flowcharts providing additional details regarding the individual steps of the method shown in FIG. 15.

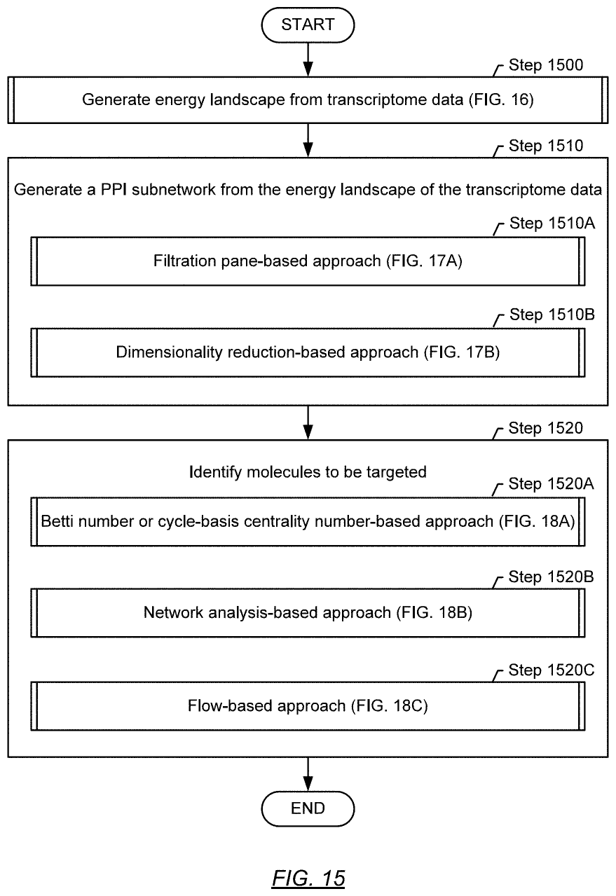

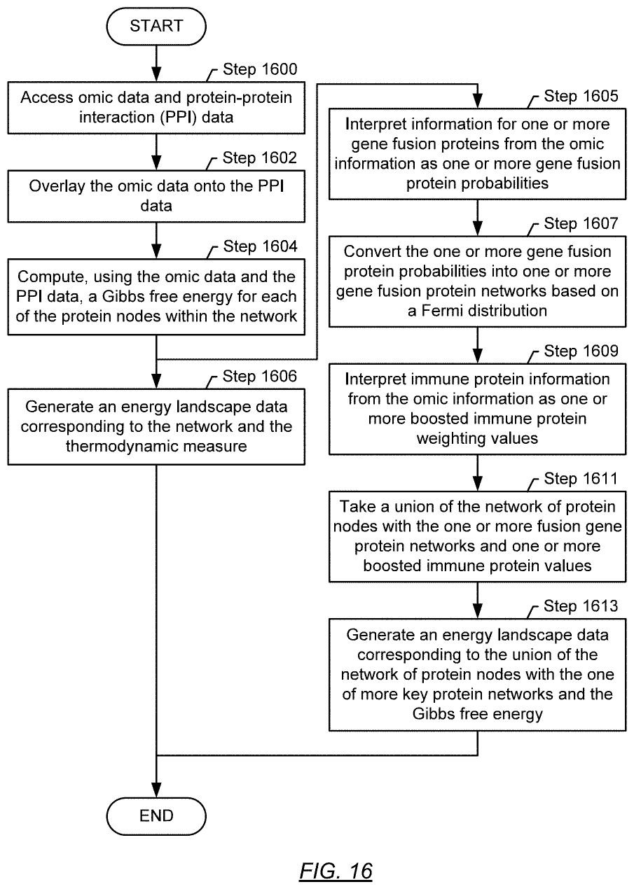

In Step 1500, an energy landscape is computed from transcriptome data. Step 1500 is described in detail in FIG. 16.

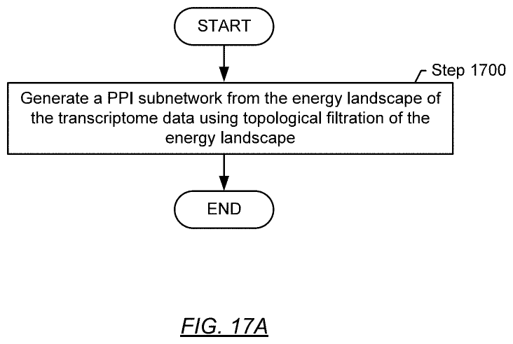

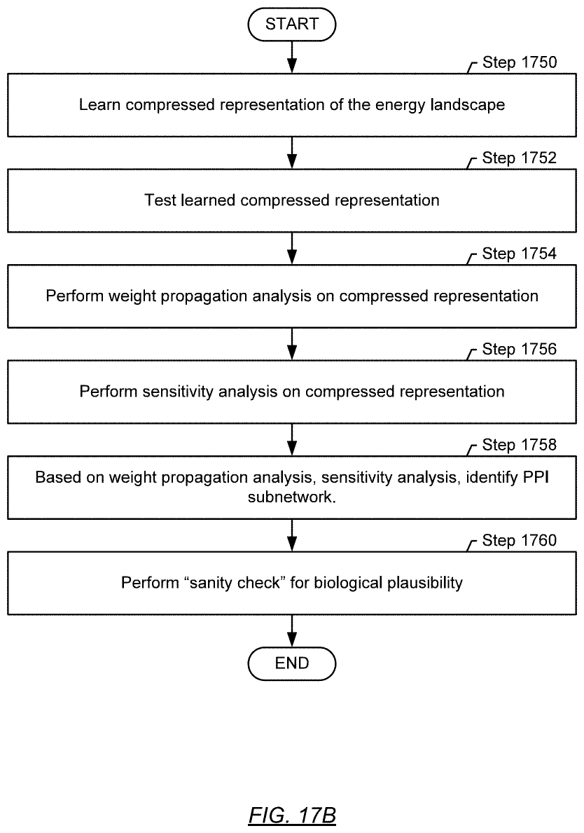

In Step 1510, a protein-protein interaction (PPI) subnetwork is computed from the energy landscape of the transcriptome data. Step 1510 may be performed to reduce the complexity associated with the energy landscape. Two alternative approaches are shown: In Step 1510A, a filtration pane-based approach is used, as described in FIG. 17A. In Step 1510B, a dimensionality reduction-based approach is used, as described in FIG. 17B.

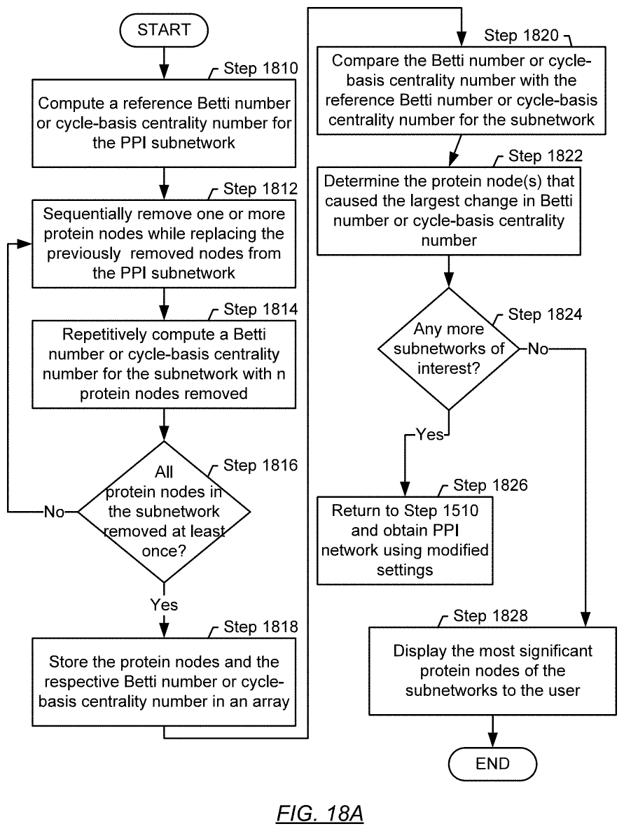

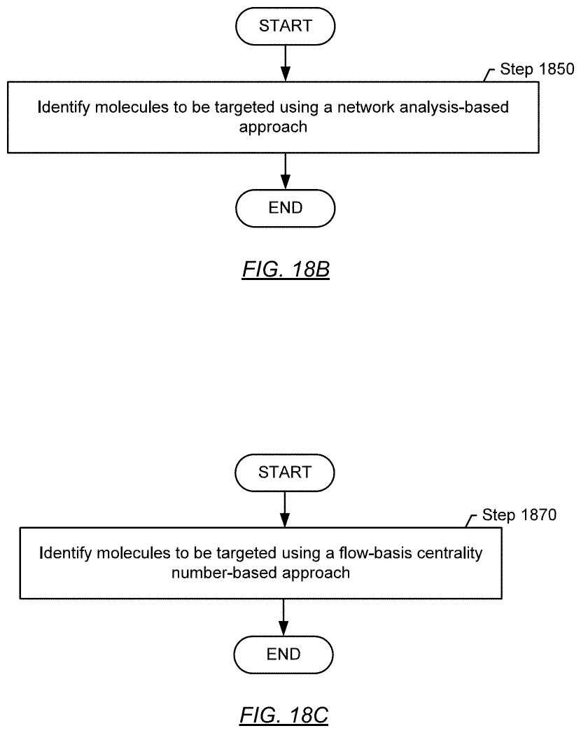

In Step 1520, molecules to be targeted are identified using the previously generated PPI subnetworks. Three alternative approaches are shown: In Step 1520A, a Betti number or cycle-basis centrality number-based approach is used, as described in FIG. 18A. In Step 1520B, a social graph theory-based approach is used, as described in FIG. 18B. In Step 1520C, a flow-based approach is used, as described in FIG. 18C.

Steps 1500, 1510 and 1520 may be all executed, or only a subset of these steps may be executed. For example, only Step 1500 may be executed to compute an energy landscape.

Turning to FIG. 16, a method for computing an energy landscape using a thermodynamic interpretation from, for example, the transcriptome data of a patient, is described.

In Step 1600, the omic data and PPI data are accessed. In one or more embodiments, the omic data is the genomic information that is derived from one or more of RNA (e.g., mRNA, rRNA, tRNA, and other non-coding RNA) transcriptome values, RNA sequencing (RNA-seq), Clustered regularly interspaced short palindromic repeats (CRISPR), and mass-spec proteomics. In one or more embodiments, the PPI data is a PPI network, such as, but is not limited to, a human PPI network data comprising a network of protein nodes.

In one or more embodiments, the omic data and the PPI data may be obtained from at least one source including an academic database, a public database, and a private database. In one or more embodiments, the omic data and the PPI data may be stored in a data repository.

In Step 1602, the omic data is overlaid onto the PPI data. In one or more embodiments each protein node within network of the PPI data is assigned its respective omic data. Once the omic data has been overlaid, the log(2) transformed transcription data is first rescaled to be in the range [0, 1]. In one or more embodiments, the most highly, positively expressed value will be set to 1.0 and the most negatively, down-regulated value will be set to 0.

It would be apparent to one of ordinary skill in the art that this is comparable to stating that the most strongly up-regulated gene produces a protein of very great concentration, relative to the most strongly down-regulated gene that will result in the lowest protein concentration.

In Step 1604, a thermodynamic measure for each of the protein nodes within the network of the PPI data is computed using the omic data. In one or more embodiments, the thermodynamic measure of each protein node is the Gibbs free energy. The Gibbs free energy is computed for each protein node by applying the rescaled value of each protein node into Eq. [1]. In one or more embodiments, the overall Gibbs free energy of the PPI data may be obtained using Eq. [2].

In Step 1606, an energy landscape data corresponding to the network and the thermodynamic measure is generated.

In one or more embodiments, the PPI data and Gibbs free energy calculations obtained in Step 1604 may be further modified to incorporate additional information in the form of Fermi energy level distributions that assign different statistical weights or energy levels to products of gene fusion that have been identified and correlated with certain disease indications. Other proteins within a PPI that may be assigned to different energy levels may include immunological proteins, and proteins associated with inflammation in various tissues. In order to incorporate this additional analysis in the workflow, methods in accordance with the present disclosure may proceed in some embodiments to Step 1605, in which information regarding one or more key proteins from the omic information generated in Step 1600 is interpreted as one or more key protein probabilities.

In particular embodiments, products of gene fusion, such as immunological proteins, and proteins associated with inflammation, may be considered and used to enhance the detail present in the energy landscape data obtained in Step 1611. For example, omic information regarding products of gene fusion and immune regulators may be obtained from the omic and PPI data at Step 1600. Step 1605 then includes the additional steps of interpreting fusion information from the omic information as one or more gene fusion probabilities, and converting the one or more gene fusion probabilities into a set gene fusion networks based on a Fermi distribution at Step 1607.

In addition to fusion proteins, other proteins may be weighted more heavily and placed on a higher level in a Fermi distribution. For example, at Step 1609, the immune regulator information may be obtained from the omic information as one or more boosted immune regulator weighting values based on a Fermi distribution. Those skilled in the art will appreciate that the described steps may be performed for any fusion protein and that an immune protein is merely provided as an example. In one or more embodiments a PPI network may be modified by one or more gene fusion protein probabilities, one or more boosted immune regulator values, or both. At Step 1611, a union of the network of protein nodes with one or both of the set of gene fusion networks and the boosted immune regulator weighting values is then obtained and used to generate an updated energy landscape at 1613.

The above-described steps of FIG. 16 may be used to construct a thermodynamically inspired energy landscape for a patient, given the patient's transcriptome data (which may include at least a list of RNA labels and a numerical value corresponding to their expression). The resulting graph may include ˜10,000 nodes (RNA corresponding to a given RNA sequencing chip used) with anywhere between 200,000-2,000,000 edges, depending on which protein-protein interaction reference graph is used. Accordingly, the resultant energy landscape is highly complex and may be difficult to use for immediate therapeutic purposes.

Additional subsequently described steps may be performed to identify or select the subnetwork of molecules that characterizes the patient's information—their molecular signature. Various methods may be used, as described with reference to FIGS. 17A and 17B.

Turning to FIG. 17A, In Step 1700, a PPI subnetwork is generated by applying a topological filtration to the energy landscape of the PPI data.

In one or more embodiments, the energy landscape contains a plurality of energy wells that are subnetworks of the PPI data. These PPI subnetworks are known as persistent homology. In one or more embodiments, the plurality of energy wells is also referred to as energetic subnetworks or Gibbs homology networks.

In one or more embodiments, the topological filtration is also referred to as a filtration threshold. The filtration threshold may be moved up from far below the lowest minima on an energy landscape. As the filtration threshold is moved up further, small connected PPI subnetworks, and later larger connected PPI subnetworks are revealed. In one or more embodiments, the filtration threshold (user set threshold) may be a value in a range of approximately 1 to 20,000.

It would be apparent to one of ordinary skill in the art that when the filtration threshold value is low, the complexity of the PPI subnetwork is also low. Similarly, when the filtration threshold value is high, the complexity of the PPI subnetwork is also high.

As an alternative to the above-described use of topological filtration, other approaches based on a dimensionality reduction may be used. These approaches may include, but are not limited to, matrix factorization techniques, statistical methods, deep learning techniques such as autoencoders and/or generative methods such as generative adversarial networks. Specifically, methods such as K-means clustering, principal component analysis, local linear embedding, independent component analysis, unsupervised dictionary learning, restricted Boltzmann machines and autoencoders may be used.

Turning to FIG. 17B, a dimensionality reduction using autoencoders is described. In Step 1750, the autoencoder may learn a compressed representation of the energy landscape. The autoencoder may operate on the PPI network data obtained in Step 1500, but may incorporate additional patient information including, but not limited to, genome/exome information, methylation information, phosphorylation information, and/or other patient omic data.

In one or more embodiments, variational or stacked denoising autoencoders are used to identify subnetworks of interest. An autoencoder is a machine learning technique that teaches a neural network to reconstruct the original input. A deep autoencoder passes the input through a bottleneck layer (typically fewer nodes than the input), and in effect learns a compressed representation of the original. A variational autoencoder adds noise from a distribution to the input, forcing the network to learn to filter out the true signal from noisy data. In this manner, a variational autoencoder taking as input the energy landscape (as obtained in Step 1500), with an input node corresponding to each RNA node, and a bottleneck layer of, for example, 100 or 500 nodes, may be used for reconstructing the original energy landscape, impervious to the added noise. For each energy landscape, the values of those 100 or 500 nodes may characterize a compressed representation of the initial 10,000 nodes (or any other number of nodes).

Subsequently, the learned compressed representation may be tested for biological plausibility as described in Steps 1752, 1754, and 1756.

In Step 1752, the learned compressed representation is tested using one or more classification tasks, to ensure biological relevance. A downstream classification task may take the compressed representation nodes as input and may be used to identify tissue of origin, and in the case of a disease such as cancer, whether the sample was malignant or benign, and in the case of malignant tumors, how long the patient lived.

In Step 1754, a weight propagation analysis is performed on the learned compressed representation. The weight propagation analysis may enable the identification of input nodes (e.g., Gibbs energy landscape molecules) that contribute the most to the bottleneck layers for a given sample.

In Step 1756, a sensitivity analysis is performed on the learned compressed representation. The sensitivity analysis may reveal, by changing the Gibbs energy of the input molecules, which of the input molecules affect the bottleneck layer the most.

The weight propagation and sensitivity analysis in combination may yield a set of input nodes that matter, thus reflecting the subnetworks of interest, from the energy landscape, as shown in Step 1758.

In Step 1760, a sanity check for biological plausibility is performed for the identified subnetworks. Biological plausibility may be assessed based on, for example, an overlap with known biological networks (such as signaling pathways, metabolic pathways, disease pathways, etc., which may be obtained from the literature, e.g., KEGG, Reactome, PantherDb, etc.).

Other methods for selecting subnetworks may be used without departing from the disclosure. These other methods include, but are not limited to, clustering to partition the initial set of nodes into small clusters; and matrix factorization or decomposition, casting the input RNA as a matrix, and with the decomposition of the matrix corresponding to subnetworks of interest.

Turning to FIG. 18A, a method for identifying molecules to be targeted using a Betti number or cycle-basis centrality number-based approach, is shown. The described method is one of a variety of possible methods based on applying topological measures to graphs.

in Step 1810, a Betti number or cycle-basis centrality number is computed for the generated PPI subnetwork. In one or more embodiments, the Betti number or cycle-basis centrality number of the PPI subnetwork is computed based on the number of rings of four or more proteins nodes within the PPI subnetwork. This Betti number or cycle-basis centrality number is used as a reference Betti number or cycle-basis centrality number.

It would be apparent to one of ordinary skill in the art that as the PPI subnetwork gets more complex, the Betti number or cycle-basis centrality number of the PPI subnetwork would also change. For example, a PPI subnetwork generated using a filtration threshold value of 10 may have a different Betti number or cycle-basis centrality number compared to a PPI subnetwork generated using a filtration threshold value of 1000.

In Step 1812, one or more protein nodes are sequentially removed from the PPI subnetwork. In one or more embodiments, when one or more protein nodes are removed, the previously removed node(s) are replaced. In one or more embodiments, the term “sequentially” is defined as following in a sequence. For example, the protein nodes in the PPI subnetwork are removed in a predetermined sequence. This ensures that all of the protein nodes in the PPI subnetwork are removed at least once.

In Step 1814, a Betti number or cycle-basis centrality number for the PPI subnetwork is repetitively computed each time one or more protein nodes are removed.

In Step 1816, a check is conducted to determine whether all of the protein nodes within the PPI subnetwork have been removed at least once. If the result of the check is NO, then Steps 1812 and Steps 1814 are repeated until all of the protein nodes in the PPI subnetwork have been removed at least once. If the result of the check is YES, then the protein nodes and the respective Betti number or cycle-basis centrality numbers are stored into an array in Step 1818.

In one or more embodiments, the array in Step 1818 maps each of the removed protein node(s) to the respective Betti number or cycle-basis centrality number computed for the PPI subnetwork with the protein node(s) removed.

In Step 1820, the recorded Betti number or cycle-basis centrality numbers are compared to the reference Betti number or cycle-basis centrality number computed in Step 1810.

Based on the results of Step 1820, the protein node(s) that caused the largest change in the Betti number or cycle-basis centrality number is determined in Step 1822. In one or more embodiments, the change in the Betti number or cycle-basis centrality number represents an effect that the protein node(s) has on a network complexity of the PPI data and the removed protein node(s) that causes a highest drop of the network complexity is the most significant molecular target(s).

In one or more embodiments, the phrase “the most significant molecular target(s)” is defined as the protein node(s) in a network or subnetwork that causes the largest change in Betti number or cycle-basis centrality number when removed. In other words, the “most significant” molecular target(s) is the number one molecular target(s) of choice when administering drugs during therapy.

In Step 1824, a determination is made whether there are other PPI subnetworks of interest. If the determination in Step 1824 results in a YES, the system returns to Step 1510 and applies a different filtration threshold value or a different parameterization of the dimensionality reduction algorithm to the PPI data to obtain a different PPI subnetwork to repeat the previously described steps for the new PPI subnetwork. If the determination in Step 1824 results in a NO, the system proceeds to Step 1828 and displays the most significant protein node(s) of the PPI subnetwork(s) to the user.

In one or more embodiments, when the complexity of the PPI subnetwork is low, removing any individual protein will drop the Betti number or cycle-basis centrality number by the same amount resulting in as many as eight or more equivalent targets. In contrast, at high complexities, there is typically only one node that leads to the biggest drop in Betti number or cycle-basis centrality number. In one or more embodiments the filtration threshold is optimized by identifying the best targets through a systematic application of thresholds between 8 and 128. For each threshold, the total Gibbs energy and the reference Betti number or cycle-basis centrality number for each PPI subnetwork is computed. In one or more embodiments, the best threshold is determined as 32.

Turning to FIG. 18B, a method for identifying molecules to be targeted using a network analysis-based approach, is shown. Network analysis is another method that is based on applying topological measures to graphs. In the context of designing a therapy, once a subnetwork of interest has been identified, molecules to be targeted need to be identified. In one embodiment of the disclosure, the PPI subnetwork is treated analogous to a social network. Accordingly, the energy landscape may be analyzed based on connectivity measures such as betweenness centrality, and other measures are of interest.

Turning to FIG. 18C, a method for identifying molecules to be targeted using a flow-basis centrality number-based approach, is shown. Network analysis is another method that is based on applying topological measures to graphs. The approach may incorporate additional information such as directionality of molecular interactions, to reflect the fact that some protein interactions only occur in one direction. Similarly, the approach may incorporate the strength of molecular interactions through the introduction of weights between nodes. A weight may quantify a protein binding affinity for an interaction. Further, a weight may quantify an energy gradient between two molecules. Based on the availability of directionality and/or weights, a flow network model may be created to, for example, identify reaction paths, or to recognize when a molecule has a redundant path in the network, etc. Subsequently, flow network theory may be used to rank molecules, by identifying the molecular paths within the network, and recognizing which underlying biological processes these paths enable, and then prioritize inhibiting those molecules which disrupt the most important paths, while minimizing any redundant paths for the processes to be disrupted.

The embodiments and examples set forth herein were presented in order to best explain the present invention and its particular application and to thereby enable those skilled in the art to make and use the invention. However, those skilled in the art will recognize that the foregoing description and examples have been presented for the purposes of illustration and example only. The description as set forth is not intended to be exhaustive or to limit the invention to the precise form disclosed. For example, while the above description discusses methods in context of human therapeutic approaches, those skilled in the art will appreciate that the described methods are equally applicable to other domains such as veterinary medicine, etc.

While the invention has been described with respect to a limited number of embodiments, those skilled in the art, having benefit of this disclosure, will appreciate that other embodiments may be devised which do not depart from the scope of the invention as disclosed herein. Accordingly, the scope of the invention should be limited only by the attached claims.

What is claimed is:

1. A method to select a molecular target for therapeutic application, comprising:
accessing omic information and protein-protein interaction (PPI) data, the PPI data comprising a network of protein nodes from at least one source;
computing a Gibbs free energy for each protein node within the network of protein nodes using the omic information and the PPI data;
interpreting information for one or more products of gene fusion from the omic information as one or more gene fusion protein probabilities;
converting the one or more gene fusion protein probabilities into one or more gene fusion protein networks based on a Fermi distribution;
taking a union of the network of protein nodes with the one or more gene fusion protein networks; and
generating an energy landscape data corresponding to the union of the network of protein nodes with the one or more gene fusion protein networks, and the Gibbs free energy.

2. The method of claim 1, further comprising generating a PPI subnetwork from the energy landscape data.

3. The method of claim 2, wherein generating the PPI subnetwork comprises applying a topological filtration to the energy landscape data.

4. The method of claim 2, wherein generating the PPI subnetwork comprises a dimensionality reduction performed on the energy landscape data.

5. The method of claim 2, further comprising identifying at least one molecule to be targeted.

6. The method of claim 5, wherein identifying the at least one molecule to be targeted comprises:
computing at least one of a first Betti number or cycle-basis centrality number for the PPI subnetwork;
sequentially removing a first protein node from the PPI subnetwork;
computing at least one of a second Betti number or cycle-basis centrality number for the PPI subnetwork with the first protein node removed;
computing a change between the first Betti number or cycle-basis centrality number and the second Betti number or cycle-basis centrality number;
replacing the first protein node into the PPI subnetwork;
sequentially removing a second protein node from the PPI subnetwork, wherein the second protein node is different from the first protein node;
computing a third Betti number or cycle-basis centrality number for the PPI subnetwork with the second protein node removed and the first protein node replaced;
computing a change between the first Betti number or cycle-basis centrality number and the third Betti number or cycle-basis centrality number; and
determining, based on the change between the first Betti number or cycle-basis centrality number and the second Betti number or cycle-basis centrality number and the change between the first Betti number or cycle-basis centrality number and the third Betti number or cycle-basis centrality number, a most significant molecular target within the PPI subnetwork.

7. The method of claim 5, wherein identifying the at least one molecule to be targeted comprises at least one selected from a group consisting of treating the PPI subnetwork analogous to a social network, and a flow network.

8. The method of claim 1, wherein converting the one or more gene fusion protein probabilities into one or more gene fusion protein networks based on a Fermi distribution comprises placing a gene fusion protein on a higher energy level of the Fermi distribution that corresponds with the respective gene fusion probability.

9. The method of claim 1, further comprising:
interpreting immune regulator information from the omic information as one or more boosted immune regulator weighting values based on a Fermi distribution;
wherein taking a union of the network of protein nodes with the one or more gene fusion protein networks further comprises: taking a union of the network of protein nodes with the one or more gene fusion protein networks and the one or more boosted immune regulator weighting values;
wherein generating an energy landscape data corresponding to the union of the network of protein nodes with the one or more gene fusion protein networks, and the Gibbs free energy further comprises: generating an energy landscape data corresponding to the union of the network of protein nodes with the one or more gene fusion protein networks and the one or more boosted immune regulator values, and the Gibbs free energy.

10. A non-transitory computer-readable medium having instructions stored thereon that, in response to execution by a computer system, cause the computer system to perform operations comprising:
accessing omic information and protein-protein interaction (PPI) data, the PPI data comprising a network of protein nodes from at least one source;
computing a Gibbs free energy for each protein node within the network of protein nodes using the omic information and the PPI data;
interpreting information for one or more products of gene fusion from the omic information as one or more gene fusion protein probabilities;
converting the one or more gene fusion protein probabilities into one or more gene fusion protein networks based on a Fermi distribution;
taking a union of the network of protein nodes with the one or more gene fusion protein networks; and
generating an energy landscape data corresponding to the union of the network of protein nodes with the one or more gene fusion protein networks, and the Gibbs free energy.

11. The non-transitory computer-readable medium of claim 10, wherein the instructions stored thereon further cause the computer system to perform operations comprising generating a PPI subnetwork from the energy landscape data.

12. The non-transitory computer-readable medium of claim 11, wherein generating the PPI subnetwork comprises applying a topological filtration to the energy landscape data.

13. The non-transitory computer-readable medium of claim 11, wherein generating the PPI subnetwork comprises a dimensionality reduction performed on the energy landscape data.

14. The non-transitory computer-readable medium of claim 11, wherein the instructions stored thereon further cause the computer system to perform operations comprising identifying at least one molecule to be targeted.

15. The non-transitory computer-readable medium of claim 14, wherein identifying the at least one molecule to be targeted comprises:
computing at least one of a first Betti number or cycle-basis centrality number for the PPI subnetwork;
sequentially removing a first protein node from the PPI subnetwork;
computing at least one of a second Betti number or cycle-basis centrality number for the PPI subnetwork with the first protein node removed;
computing a change between the first Betti number or cycle-basis centrality number and the second Betti number or cycle-basis centrality number;
replacing the first protein node into the PPI subnetwork;
sequentially removing a second protein node from the PPI subnetwork, wherein the second protein node is different from the first protein node;
computing a third Betti number or cycle-basis centrality number for the PPI subnetwork with the second protein node removed and the first protein node replaced;
computing a change between the first Betti number or cycle-basis centrality number and the third Betti number or cycle-basis centrality number; and
determining, based on the change between the first Betti number or cycle-basis centrality number and the second Betti number or cycle-basis centrality number and the change between the first Betti number or cycle-basis centrality number and the third Betti number or cycle-basis centrality number, a most significant molecular target within the PPI subnetwork.
